(19) United States
(12) Patent Application Publication
(10) Pub. No.: US 2025/0360230 A1
(43) Pub. Date: Nov. 27, 2025

(54) MINIGENE THERAPY

(71) Applicant: University of Massachusetts, Westborough, MA (US)

(72) Inventors: Guangping Gao, Worcester, MA (US); Hemant Khanna, Worcester, MA (US)

(73) Assignee: University of Massachusetts, Westborough, MA (US)

(21) Appl. No.: 19/269,517

(22) Filed: Jul. 15, 2025

Related U.S. Application Data
(62) Division of application No. 17/612,653, filed on Nov. 19, 2021, now Pat. No. 12,390,539.
(60) Provisional application No. 62/850,405, filed on May 20, 2019.
(60) Provisional application No. 62/899,601, filed on Sep. 12, 2019.
(60) Provisional application No. 62/967,521, filed on Jan. 29, 2020.

(51) Int. Cl. A61K 48/00; A61K 35/76; A61P 27/02; C07K 14/705; C12N 5/079; C12N 7/00; C12N 15/113; C12N 15/86

(52) U.S. Cl. CPC A61K 48/0058; A61K 35/76; A61P 27/02; C07K 14/705; C12N 5/0621; C12N 7/00; C12N 15/113; C12N 15/86; C12N 2310/10; C12N 2750/14122; C12N 2750/14142; C12N 2830/008

(57) ABSTRACT

Aspects of the disclosure relate to compositions and methods useful for treating ocular ciliopathies, for example Leber congenital amaurosis (LCA). In some embodiments, the disclosure provides isolated nucleic acids comprising a transgene encoding a CEP290 protein fragment, and methods of treating ocular ciliopathies using the same.

### RELATED APPLICATIONS

This application is a divisional of U.S. application Ser. No. 17/612,653, filed Nov. 19, 2021, which is a national stage filing under 35 U.S.C. 371 of international PCT Application PCT/US2020/033600, filed May 19, 2020, which claims the benefit under 35 U.S.C. § 119(e) of the filing date of U.S. provisional Application Ser. Nos. 62/967,521, filed Jan. 29, 2020, 62/850,405, filed May 20, 2019, and 62/899,601, filed Sep. 12, 2019, the entire contents of each application which are incorporated herein by reference.

### GOVERNMENT SUPPORT

This invention was made with government support under EY022372, EY029050, NS076991, AI100263, and HL131471, awarded by the National Institutes of Health. The government has certain rights in the invention.

### REFERENCE TO AN ELECTRONIC SEQUENCE LISTING

The content of the electronic sequence listing (U012070124US04-SEQ-SCC.xml; Size: 105,613 bytes; and Date of Creation: Jul. 7, 2025) is herein incorporated by reference in its entirety.

### BACKGROUND

Ciliopathies represent a group of diseases and disorders characterized by abnormal cilial formation or function. For example ocular ciliopathies may lead to retinal degeneration, reduced visual acuity, and/or blindness. CEP290-associated Leber congenital amaurosis (LCA) is one of the most common and severe forms of retinal degenerative diseases. However, no treatment or cure currently exists. Generally, the large size of cilia-associated genes, for example the CEP290 gene (˜8 kb), has limited the development of successful therapy using conventional Adeno-associated Viral (AAV) vector-mediated gene delivery approaches because the cargo size exceeds the ˜4700 bp packaging limit of rAAVs. Use of genome editing (such as CRISPR/Cas9 approach) and antisense oligonucleotides can have off-target effects and are typically applicable to only one type of mutation in a cilia-associated gene. Accordingly, novel compositions and methods for treating ciliopathies are needed.

### SUMMARY

Aspects of the disclosure relate to compositions and methods useful for delivering minigenes to a subject. Accordingly, the disclosure is based, in part, on gene therapy vectors, such as viral (e.g., rAAV) vectors, comprising one or more gene fragments encoding a therapeutic gene product, such as a protein or peptide (e.g., a minigene). In some aspects, a gene therapy vector further comprises one or more inhibitory nucleic acids that target an endogenous gene variant (e.g., mutant) that is associated with a disease or disorder (e.g., a gene associated with a ciliopathy). In some embodiments, the one or more inhibitory nucleic acids do not silence gene expression of the gene product encoded by the minigene. In some embodiments, methods are provided for treating ciliopathies (e.g., ocular ciliopathies), for example disorders and diseases characterized by a mutation or deletion of a cilia-associated gene, such as the CEP290 gene which is associated with Leber congenital amaurosis (LCA).

Accordingly, in some aspects, the disclosure relates to an isolated nucleic acid comprising a transgene encoding a CEP290 fragment having the amino acid sequence set forth in any one of SEQ ID NOs: 10-19 or 36.

In some embodiments, a CEP290 fragment is encoded by a nucleic acid having the sequence set forth in any one of SEQ ID NOs: 20-29 and 34-35. In some embodiments, a CEP290 fragment is encoded by a nucleic acid having the sequence set forth in SEQ ID NO: 29 or 34. In some embodiments, a nucleic acid encodes a CEP290 protein fragment comprising amino acids 1-200 and 580-1180 of a human CEP290. In some embodiments, a nucleic acid encodes a CEP290 protein fragment comprising amino acids 1-200 and 580-1180 of SEQ ID NO: 1. In some embodiments, a nucleic acid encodes a CEP290 protein fragment comprising or consisting of the amino acid sequence set forth in SEQ ID NO: 19.

In some embodiments, a CEP290 fragment is encoded by a nucleic acid having the sequence set forth in SEQ ID NO: 35. In some embodiments, a nucleic acid encodes a CEP290 protein fragment comprising amino acids 1-380 and 580-1180 of a human CEP290. In some embodiments, a nucleic acid encodes a CEP290 protein fragment comprising amino acids 1-380 and 580-1180 of SEQ ID NO: 1. In some embodiments, a nucleic acid encodes a CEP290 protein fragment comprising or consisting of the amino acid sequence set forth in SEQ ID NO: 36.

In some embodiments, a transgene further comprises a promoter. In some embodiments, a promoter is a CB6 promoter, a CBA promoter, or a tissue-specific promoter. In some embodiments, a tissue specific promoter is an eye-specific promoter. In some embodiments, an eye-specific promoter is a retinoschisin promoter, K12 promoter, a rhodopsin promoter, a rod-specific promoter, a cone-specific promoter, a rhodopsin kinase promoter (e.g., a GRK1 promoter), or an interphotoreceptor retinoid-binding protein proximal (IRBP) promoter.

In some embodiments, a transgene further comprises an intron (e.g., a chicken-beta actin intron, a synthetic intron, MBL intron, etc.). In some embodiments, the intron is positioned between the promoter and minigene (e.g. MiniCEP290) coding sequence of the transgene.

In some embodiments, a transgene is flanked by adeno-associated virus (AAV) inverted terminal repeats (ITRs). In some embodiments, AAV ITRs are AAV2 ITRs or a variant thereof, such as ΔITR or mTR ITRs.

In some aspects, the disclosure provides a vector comprising an isolated nucleic acid as described herein. In some embodiments, a vector is a plasmid.

In some aspects, the disclosure relates to a host cell comprising an isolated nucleic acid or a vector as described herein.

In some embodiments, the disclosure provides a recombinant adeno-associated virus (rAAV) comprising: a capsid protein; and, an isolated nucleic acid as described herein.

In some embodiments, a capsid protein is AAV8 capsid protein or AAV5 capsid protein. In some embodiments, a capsid protein is an AAV8 capsid protein or a variant thereof. In some embodiments, a capsid protein is an AAV5 capsid protein or a variant thereof. In some embodiments, a capsid protein comprises the amino acid sequence set forth in SEQ ID NO: 9.

In some embodiments, an rAAV is a self-complementary AAV (scAAV).

In some embodiments, an rAAV is formulated for delivery to the eye. In some embodiments, an rAAV is formulated for subretinal delivery. In some embodiments, an rAAV comprises one or more of the CEP290 fragments described by the disclosure and an AAV8 capsid protein or AAV5 capsid protein. In some embodiments, an rAAV comprises (i) a nucleic acid sequence encoding a CEP290 fragment comprising the amino acid sequence set forth in SEQ ID NO: 19 or 34 operably linked to a rhodopsin kinase (RK) promoter; and (ii) an rAAV8 capsid protein or AAV5 capsid protein. In some embodiments, the rAAV is formulated for subretinal delivery.

In some aspects, the disclosure provides a composition comprising an rAAV as described herein, and a pharmaceutically acceptable excipient. In some embodiments, a composition comprises a plurality of the rAAVs. In some embodiments, each rAAV of a plurality encodes a different CEP290 fragment.

In some aspects, the disclosure provides a method for treating an ocular ciliopathy in a subject in need thereof, the method comprising administering to a subject having an ocular ciliopathy a therapeutically effective amount of an isolated nucleic acid, rAAV, or composition as described herein.

In some embodiments, an ocular ciliopathy is associated with a mutation of the CEP290 gene in the subject or a deletion of a CEP290 gene in a subject. In some embodiments, a mutation in a CEP290 gene is an intronic mutation, a nonsense mutation, a frameshift mutation, a missense mutation, or any combination thereof. In some embodiments, the mutation or deletion of CEP290 results in retinal degeneration, photoreceptor degeneration, retinal dysfunction, and/or loss of vision.

In some embodiments, an ocular ciliopathy is Leber congenital amaurosis (LCA), Joubert syndrome, Bardet-Biedl syndrome, Meckel syndrome, Usher syndrome, Nephronophthisis, or Senior-Løken syndrome. In some embodiments, the ocular ciliopathy is Leber congenital amaurosis (LCA). In some embodiments such as Retinitis Pigmentosa (RP), the severity of an ocular ciliopathy is modified by CEP290.

In some embodiments, a subject is a human characterized by one or more CEP290 mutations (e.g., one or more mutations in a CEP290 gene) that occurs at position c.2991+1655. In some embodiments, at least one mutation is A1655G.

In some embodiments, administration of an isolated nucleic acid, rAAV, or composition results in delivery of a CEP290 fragment (e.g., a transgene encoding a CEP290 fragment) to the eye of a subject. In some embodiments, administration is via injection. In some embodiments, injection comprises subretinal injection or intravitreal injection. In some embodiments, administration is topical administration to the eye of the subject. In some embodiments, administration is by subretinal administration.

In some embodiments, an effective amount (e.g., administration of an effective amount of an isolated nucleic acid (messenger RNA), isolated CEP290 fragment protein, rAAV, or composition) results in photoreceptor (PR) function (e.g., increased PR function as measured by ERG). In some embodiments, an effective amount (e.g., administration of an effective amount of an isolated nucleic acid (messenger RNA), isolated CEP290 fragment protein, rAAV, or composition) results in photoreceptor (PR) function (e.g., increased PR function as measured by ERG), for up to fourteen weeks.

### BRIEF DESCRIPTION OF DRAWINGS

FIG. 1 shows schematic depiction of the full-length CEP290 gene representing the locations of distinct protein interaction domains.

FIG. 2A shows microscopy data relating to cilial number and length in mouse embryonic fibroblasts (MEFs) from wild-type (WT) and Cep290^{rd16} mice that were serum-starved for 24 h (for cilia growth) and then stained with anti-acetylated α-tubulin antibody (cilia marker). The lower images depict higher magnification of cilia. FIG. 2B shows a statistically significant decrease in the length of cilia in mutant MEFs.

FIG. 3 shows Cep290^{rd16} MEFs transfected with constructs encoding GFP or GFP-CEP290, followed by staining with ARL13b (cilia marker) and γ-tubulin. A significant increase in cilia length of cells expressing full-length CEP290 was observed. GFP-encoding construct was used as negative control. **: p<0.001.

FIG. 4A shows a schematic representation of the human CEP290 protein and deleted variants. Myo-tail: Myosin tail homology domain. Additional protein-interaction domains are also not shown. FIG. 4B shows immunoblot analysis using anti-GFP antibody of mouse fibroblasts transiently transfected with the constructs described in FIG. 4A. Specific protein bands (depicted by arrows) were detected indicating that the deleted variants are stably expressed in cells.

FIG. 5A shows immunostaining of Cep290^{rd16} fibroblasts transiently transfected with plasmid encoding GFP-fused full-length (FL) CEP290 and indicated variants with GFP, γ-tubulin, ARL13B antibodies. Nuclei were stained with DAPI. Longer arrows indicate basal body/ciliary localization of the proteins whereas shorter arrows mark the diffuse staining. FIG. 5B shows cilia length of cells (n>200) described in FIG. 5A quantified using ImageJ. *: p<0.001. ns: not significant.

FIG. 6A shows immunoblot analysis of Cep290^{rd16} fibroblasts transiently transfected with plasmid encoding GFP alone or GFP-fused indicated variants, using anti-GFP antibody. Arrows point to the expected size protein product. Molecular mass marker is shown in kDa. FIG. 6B shows immunostaining of the cells using GFP and ARL13B (cilia marker) antibodies. Nuclei were stained with DAPI. Arrows indicate basal body/ciliary localization of the proteins. FIG. 6C shows the cilia length of the cells (n>200) quantified using ImageJ. *: p<0.001.

FIGS. 7A-7B show in vivo physiological rescue potential of miniCEP290⁵⁸⁰⁻¹¹⁸⁰. FIG. 7A shows Cep290^{rd16} mice subretinally injected at P0/P1 stage with indicated miniCEP290s or GFP, and analyzed by ERG at 3 weeks post injection. Age-matched uninjected WT or Cep290^{rd16} (littermates) mice were used as controls for ERG. The ERG a-wave is represented by arrows while b-wave vis depicted using arrowheads. Data represent analysis of at least 6 mice. ***: p<0.0001; ns: not significant. FIG. 7B shows scotopic a-wave and b-wave amplitude for mice subretinally injected at P0/P1 stage with indicated miniCEP290s or GFP, and analyzed by ERG at 3 weeks post injection.

FIGS. 8A-8D show in vivo morphological rescue of photoreceptors by miniCEP290⁵⁸⁰⁻¹¹⁸⁰. FIG. 8A shows Cep290^{rd16} retinas injected with indicated miniCEP290⁵⁸⁰⁻¹¹⁸⁰ or GFP stained with DAPI. FIG. 8B shows Cep290^{rd16} retinas injected with indicated miniCEP290⁵⁸⁰⁻¹¹⁸⁰or GFP assessed by ultrathin sectioning. ONL (outer nuclear layer) is marked with vertical lines. WT retinal section is shown for comparison. INL: inner nuclear layer. FIG. 8C shows improved expression of RDS detected in the miniCEP290⁵⁸⁰⁻¹¹⁸⁰ injected Cep290^{rd16} mice. GFP staining marks the injected regions. FIG. 8D shows retinal cryosections of Cep290^{rd16} mice injected with the indicated miniCEP290s were stained with GFP (injected regions), rhodopsin (RHO; rod-specific; or M-opsin (MOP; cone-specific) antibodies and DAPI (nuclei). Outer segment (OS)-enriched opsin staining is detected in the miniCEP290⁵⁸⁰⁻¹¹⁸⁰-injected retinas. Dramatically reduced expression of opsins is detected in the miniCEP290²⁰³⁷⁻²⁴⁷⁹-injected retinas. ONL: outer nuclear layer; INL: inner nuclear layer; GCL: ganglion cell layer.

FIG. 9 shows additional embodiments of CEP290 minigenes.

FIG. 10 shows the cilia length of Cep290^{rd16} fibroblasts transiently transfected with plasmid encoding GFP alone or GFP-fused indicated variants, using anti-GFP antibody (n>200) quantified using ImageJ.

FIG. 11 shows Cep290^{rd16} mice subretinally injected at P0/P1 stage with indicated miniCEP290s or GFP, and analyzed by ERG at 3 weeks post injection. Age-matched uninjected WT or Cep290^{rd16} (littermates) mice were used as controls for ERG. The ERG a-wave is represented by arrows while b-wave vis depicted using arrowheads. Data represent analysis of at least 6 mice. ***: p<0.0001; ns: not significant.

FIG. 12 shows scotopic a-wave and b-wave amplitude for mice subretinally injected at P0/P1 stage with indicated miniCEP290s or GFP, and analyzed by ERG at 3 weeks post injection Scotopic (a- and b-waves) and photopic b-wave analysis of the injected mice performed at 4 and 5 weeks post injection and compared to the ERG at 3 weeks are shown. Age-matched uninjected WT and GFP-injected Cep290^{rd16} mice were used as controls.

FIG. 13 shows scotopic a-wave and b-wave amplitude for 10-day old mice subretinally injected with the indicated miniCEP290 construct (GRK-580-1180). ERG were recorded at the indicated days after injection.

FIG. 14 shows photopic a-wave and b-wave amplitude for 10-day old mice subretinally injected with the indicated miniCEP290 construct (GRK-580-1180). ERG were recorded at the indicated days after injection.

FIG. 15 shows micrographs of rd16 mouse embryonic fibroblasts transiently transfected with cDNA encoding the indicated minigenes and GFP.

FIG. 16 shows data for cilia length measurement of rd16 mouse embryonic fibroblasts transiently transfected with cDNA encoding the indicated minigenes encoding the protein and GFP.

FIGS. 17A-17D show scotopic and photopic wave amplitudes of CB6-promoter driving the indicated minigenes were subretinally injected into 10 day old rd16 mice. FIG. 17A shows scotopic a-wave amplitude. FIG. 17B shows photopic b-wave amplitude. FIG. 17C shows scotopic b-wave amplitude. FIG. 17D shows ERG of rd16 mice injected with CB6-1-200-580-1180 miniCEP290. ERG were recorded at indicated days after injection and compared to others.

FIG. 18 is a schematic depicting additional CEP290 minigenes.

FIG. 19 shows representative data for ERG analysis of Rhodopsin Kinase (RK) promoter-driven expression of miniCEP290-580-1180 in subretinally-injected mice. Rescue effect lasted more than 10 weeks post-injection.

FIG. 20 shows representative data for ERG analysis of Rhodopsin Kinase (RK) promoter-driven expression of miniCEP290-1181-2479 in subretinally-injected mice. Rescue effect lasted up to 8.5 weeks post-injection.

FIG. 21 shows representative data for ERG analysis of Rhodopsin Kinase (RK) promoter-driven expression of miniCEP290-580-1180/1800-2479 in subretinally-injected mice. Rescue effect lasted up to 5.5 weeks post-injection.

FIG. 22 shows representative data for ERG analysis of Rhodopsin Kinase (RK) promoter-driven expression of miniCEP290-1181-1695/1966-2479 in subretinally-injected mice.

FIG. 23 shows representative data for ERG analysis of Rhodopsin Kinase (RK) promoter-driven expression of miniCEP290-1-200/580-1180 in subretinally-injected mice. Rescue effect lasted more than 14 weeks post-injection.

FIGS. 24A-24C show data from a morphological analysis of Rhodopsin Kinase (RK) promoter driving miniCEP290-1-200/580-1180: the minigene was subretinally delivered with AAV8 at P10 stage to Cep290^{rd16} mice. The analysis was performed at 9 weeks of age. FIG. 24A shows miniCEP290-1-200/580-1180 immunofluorescence analysis of the injected retinal region (GFP; longer arrows). FIG. 24B shows data indicating improvement in rhodopsin (longer arrows) after delivery of miniCEP290-1-200/580-1180. Shorter arrows do not show GFP expression (non-transduced) and consequently exhibit undetectable rhodopsin expression. FIG. 24C shows nuclear staining data indicating more nuclear layers in the outer nuclear layer (ONL) region in the miniCEP290-1-200/580-1180-transduced area (longer arrows and longer vertical bars) as compared to the untransduced region (shorter arrow and shorter vertical bar).

FIGS. 25A-25C show codon optimized miniCEP290 constructs. FIG. 25A shows codon optimized miniCEP290 (1-200/580-1180) constructs with different promoters and introns. FIG. 25B shows codon optimized miniCEP290 (1-380/580-1180) constructs with different promoters and introns. FIG. 25C shows representative data for codon optimized miniCEP290 (1-200/580-1180) constructs packaged into AAV5 capsid and injected subretinally into mice at P10 stage. The mice were then analyzed by ERG (both scotopic and photopic) at the ages indicated.

### DETAILED DESCRIPTION

In some aspects, the disclosure relates to compositions and methods useful for treating certain genetic diseases, for example monogenic diseases, ciliopathies, etc. Monogenic diseases are diseases that are diseases that result from abnormal expression or function of a single allele of a gene. Examples of monogenic diseases include but are not limited to thalassemia, sickle cell anemia, hemophilia, cystic fibrosis, Tay Sachs disease, Fragile X syndrome, Huntington's disease, etc. Ciliopathies are genetic disorders that affect the expression or function of cellular cilia, for example ocular ciliopahies. Examples of ciliopathies include but are not limited to Alstrom syndrome, Bardet-Biedl syndrome, Joubert syndrome, Merckel syndrome, nephronophthisis, orofaciodigital syndrome, Senior-Locken syndrome, polycystic kidney disease, primary ciliary dyskinesia, and situs inversus.

The disclosure is based, in part, on isolated nucleic acids, vectors (e.g., plasmids, bacmids, etc.), and gene therapy vectors, such as viral (e.g., rAAV) vectors, comprising one or more gene fragments encoding a therapeutic gene product, such as a protein or peptide (e.g., a minigene), and optionally one or more inhibitory nucleic acids that target an endogenous gene variant (e.g., mutant) that is associated with a disease or disorder (e.g., a gene associated with a ciliopathy).

A gene therapy vector may be a viral vector (e.g., a lentiviral vector, an adeno-associated virus vector, etc.), a plasmid, a closed-ended DNA (e.g., ceDNA), etc. In some embodiments, a gene therapy vector is a viral vector. In some embodiments, an expression cassette encoding a minigene is flanked by one or more viral replication sequences, for example lentiviral long terminal repeats (LTRs) or adeno-associated virus (AAV) inverted terminal repeats (ITRS).

As used herein, “minigene” refers to an isolated nucleic acid sequence encoding a recombinant peptide or protein where one or more non-essential elements of the corresponding gene encoding the naturally-occurring peptide or protein have been removed and where the peptide or protein encoded by the minigene retains function of the corresponding naturally-occurring peptide or protein. A “therapeutic minigene” refers to a minigene encoding a peptide or protein useful for treatment of a genetic disease, for example, human centrosomal protein 290 (CEP290), dystrophin, dysferlin, Factor VIII, Amyloid precursor protein (APP), Tyrosinase (Tyr), etc. Minigenes are known in the art and are described, for example by Karpati and Acsadi (1994) Clin Invest Med 17(5):499-509; Plantier et al. (2001) Thromb Haemost. 86(2):596-603; and Xiao et al. (2007) World J. Gastroenterol. 13(2):244-9.

Generally, an isolated nucleic acid encoding a minigene (e.g., a therapeutic minigene) is between about 10% and about 99% (e.g., about 10%, about 15%, about 20%, about 25%, about 30%, about 40% about 50%, about 60%, about 70%, about 75%, about 80%, about 90%, about 99%, etc.) truncated with respect to a nucleic acid sequence encoding the corresponding naturally-occurring wild-type protein. For example, in some embodiments, a minigene encoding a CEP290 protein fragment is about 76% truncated (e.g., comprises about 24% of the nucleic acid sequence) compared to a wild-type CEP290 gene.

Aspects of the disclosure relate to isolated nucleic acids comprising a transgene encoding one or more CEP290 fragments. A “fragment” refers to a protein encoded by at least two discontinuous nucleotide sequence portions that are in frame with each other and encode a functional protein. A CEP290 fragment may comprise an amino acid sequence corresponding to one or more domains of a CEP290 protein (e.g., SEQ ID NO: 1) or portions thereof, for example one or more of a CP110-binding domain (or a portion thereof), NPHP5-binding domain (or a portion thereof), RAB8A-binding domain (or a portion thereof), a microtubule (MT) binding domain (or a portion thereof), and a RPGR binding domain (or a portion thereof). In some embodiments, a CP110-binding domain corresponds to amino acid positions 1-579 of a wild-type CEP290 protein (e.g., SEQ ID NO: 1). In some embodiments, a NPHP5-binding domain corresponds to amino acid positions 580-880 of a wild-type CEP290 protein (e.g., SEQ ID NO: 1). In some embodiments, a RAB8A-binding domain corresponds to amino acid positions 580-1695 of a wild-type CEP290 protein (e.g., SEQ ID NO: 1). In some embodiments, a MT-binding domain corresponds to amino acid positions 1696-1966 of a wild-type CEP290 protein (e.g., SEQ ID NO: 1). In some embodiments, a RPGR-binding domain corresponds to amino acid positions 1966 to 2479 of a wild-type CEP290 protein (e.g., SEQ ID NO: 1).

In some embodiments, an isolated nucleic acid encodes a CEP290 fragment comprising the amino acid sequence set forth in any one of SEQ ID NOs: 10-19 and 36. In some embodiments, an isolated nucleic acid comprises the nucleic acid sequence set forth in any one of SEQ ID NOs: 20-29 and 34-35. In some embodiments, an isolated nucleic acid comprises a nucleic acid sequence that is at least 70%, 80%, 90%, 95%, or 99% identical to the nucleic acid sequence set forth in any one of SEQ ID NOs: 20-29 and 34-35.

In some embodiments, the nucleic acid encodes a CEP290 protein fragment corresponding to amino acids 1-200 and 580-1180 of human CEP290. In some embodiments, the nucleic acid encodes a CEP290 fragment comprising amino acids 1-200 and 580-1180 of SEQ ID NO: 1. In some embodiments, an isolated nucleic acid comprises the nucleic acid sequence set forth in SEQ ID NO: 29 or 34. In some embodiments, the nucleic acid encodes a CEP290 fragment corresponding to the amino acid sequence as set forth in SEQ ID NO: 19.

In some embodiments, the nucleic acid encodes a CEP290 protein fragment corresponding to amino acids 1-380 and 580-1180 of human CEP290. In some embodiments, the nucleic acid encodes a CEP290 fragment comprising amino acids 1-380 and 580-1180 of SEQ ID NO: 1. In some embodiments, an isolated nucleic acid comprises the nucleic acid sequence set forth in SEQ ID NO: 35. In some embodiments, the nucleic acid encodes a CEP290 fragment corresponding to the amino acid sequence as set forth in SEQ ID NO: 36.

In some embodiments, a nucleic acid sequence encoding a CEP290 fragment is codon-optimized. In some embodiments a codon-optimized CEP290 fragment is encoded by the nucleic acid sequence set forth in SEQ ID NO: 34 or 35. In some embodiments, a codon-optimized nucleic acid sequence encodes a CEP290 minigene comprising the amino acid sequence set forth in SEQ ID NO: 19 or 36.

In some embodiments, a nucleic acid comprises an expression cassette comprising the sequence set forth in SEQ ID NO: 29 or 34 (e.g., a nucleic acid sequence encoding the amino acid sequence set forth in SEQ ID NO: 19) operably linked to a promoter (e.g., a rhodopsin kinase (RK) promoter). In some embodiments, the expression cassette is flanked by adeno-associated virus (AAV) inverted terminal repeats (ITRs). In some embodiments, the ITRs are AAV2 ITRs. In some embodiments, the nucleic acid is encapsidated by one or more AAV capsid proteins. In some embodiments, the one or more AAV capsid proteins are AAV8 or AAV5 capsid proteins.

In some aspects, the disclosure relates to an isolated nucleic acids (e.g., vectors, such as viral vectors) comprising an expression cassette comprising a first isolated nucleic acid sequence encoding a therapeutic minigene and a second isolated nucleic acid sequence encoding one or more inhibitory nucleic acids, wherein the expression cassette is flanked by viral replication sequences, and wherein the one or more inhibitory nucleic acids do not bind to the isolated nucleic acid encoding the therapeutic minigene.

In some aspects, the disclosure relates to AAV-mediated delivery of CEP290 gene fragments (e.g. encoding CEP290 protein fragments) lacking the “M region” to cells (e.g., ocular cells) of a subject having a disease or disorder characterized by a mutation or deletion of the CEP290 gene, which restores or improves cilial length and rescues or improves photoreceptor function. This discovery is surprising in view of previous disclosures, for example US 2016/0185832, which describes that the “M region” of the CEP290 gene is necessary to mediate microtubule localization and cilium formation. In some embodiments, the Examples section of this disclosure describes domains (e.g., fragments) of CEP290 protein that retain function in photoreceptors and can be delivered using the conventional AAV vectors.

Accordingly, in some aspects, the disclosure provides an isolated nucleic acid comprising: a first region comprising a first adeno-associated virus (AAV) inverted terminal repeat (ITR), or a variant thereof; and, a second region comprising a transgene encoding a CEP290 protein fragment, wherein the CEP290 protein fragment does not comprise amino acid positions 1695 to 1966 of SEQ ID NO: 1.

In some aspects, the disclosure provides an isolated nucleic acid comprising: a first region comprising a first adeno-associated virus (AAV) inverted terminal repeat (ITR), or a variant thereof; and, a second region comprising a transgene encoding a CEP290 protein fragment, wherein the CEP290 protein fragment comprises at least 500 contiguous amino acids of SEQ ID NO: 1. In some embodiments, the at least 500 contiguous amino acids comprises or consists of a sequence selected from SEQ ID NOs: 2, 3 and 4.

In some embodiments, the second region does not comprise amino acid positions 1695 to 1966 of SEQ ID NO: 1. In some embodiments, the transgene comprises no more than 1120 contiguous amino acids of SEQ ID NO: 1.

In some embodiments, the transgene comprises amino acid positions 580 to 1695 of SEQ ID NO: 1. In some embodiments, the CEP290 protein fragment encoded by the transgene comprises a sequence set forth in SEQ ID NO: 2. In some embodiments, the CEP290 protein fragment encoded by the transgene comprises amino acid positions 580 to 1180 of SEQ ID NO: 1, or amino acid positions 1181 to 1695 of SEQ ID NO: 1. In some embodiments, the CEP290 protein fragment encoded by the transgene comprises or consists of a sequence set forth in SEQ ID NO: 3 or 4. In some embodiments, the CEP290 protein fragment encoded by the transgene comprises (or consists of) amino acid positions 1 to 200 of SEQ ID NO: 1 and amino acid positions 580 to 1180 of SEQ ID NO: 1. In some embodiments, the CEP290 protein fragment encoded by the transgene comprises or consists of a sequence set forth in SEQ ID NO: 29 or 34. It should be appreciated that CEP290 protein fragments delivered by the transgene may be translated as a single fusion protein comprising two or more fragments, or as separate polypeptides.

In some embodiments, the transgene comprises or consists of a nucleic acid sequence selected from SEQ ID NO: 5, 6 and 7.

In some embodiments, a gene therapy vector further comprises one or more inhibitory nucleic acids that do not silence gene expression of the gene product encoded by the minigene but do silence gene expression of an endogenous protein corresponding to a wild-type or disease-associated variant of the protein encoded by the minigene. For example, in some embodiments, a gene therapy vector comprises a minigene encoding a CEP290 protein fragment and one or more inhibitory nucleic acids (e.g., dsRNA, siRNA, shRNA, miRNA, amiRNA, etc.) that inhibit expression of endogenously expressed CEP290 (e.g., a CEP290 mutant selected from c.2991+1655A>G, c.2249T>G, c.7341dupA, c.2118_2122dupTCAGG, c.3814C>T, c.679_680delGA, c.265dupA, c.180+1G?T, c.1550delT, c.4115_4116delTA, c.4966G>T, and c.5813_5817delCTTTA) but do not inhibit expression of the CEP290 fragment encoded by the minigene. The skilled artisan will also appreciate that, in some embodiments, one or more inhibitory nucleic acids that that inhibit expression of endogenously expressed CEP290 but do not inhibit expression of the CEP290 fragment encoded by the minigene may be administered to a subject in a manner that is separate from the gene therapy construct.

In some aspects, the CEP290 fragment is encoded by the messenger RNA. In other aspects, the CEP290 fragment is the protein delivered to the affected cells. In some embodiments one or more CEP290 fragments is delivered to affected cells by a nanoparticle or microsphere-based delivery system. In some embodiments, a nanoparticle or microsphere-based delivery system is formulated to penetrate the affected cell, for example via inclusion of a cell permeable peptide (cpp) sequence to the CEP290 fragment(s) or delivery system (e.g., nanoparticle).

### Methods for Treating Ocular Ciliopathies

Aspects of the invention relate to certain protein-encoding transgenes (e.g., fragments of human CEP290) that when delivered to a subject are effective for promoting growth of ocular cilia (e.g., cilia of photoreceptors) and rescue of photoreceptor structure and function in the subject. Accordingly, methods and compositions described by the disclosure are useful, in some embodiments, for the treatment of ocular ciliopathies associated with mutations or deletions of CEP290 gene, such as Leber congenital amaurosis (LCA), Joubert syndrome, Bardet-Biedl syndrome, Meckel syndrome, Usher syndrome, and Senior-Løken syndrome.

As used herein “treat” or “treating” refers to (a) preventing or delaying onset of ocular ciliopathies associated with mutations or deletions of CEP290 gene (such as Leber congenital amaurosis (LCA), Joubert syndrome, Bardet-Biedl syndrome, Meckel syndrome, Usher syndrome, or Senior-Løken syndrome); (b) reducing severity of ocular ciliopathies associated with mutations or deletions of CEP290 gene; (c) reducing or preventing development of symptoms characteristic of ocular ciliopathies associated with mutations or deletions of CEP290 gene; (d) and/or preventing worsening of symptoms characteristic of ocular ciliopathies associated with mutations or deletions of CEP290 gene. Signs and symptoms of ocular ciliopathies associated with mutations or deletions of CEP290 gene include, for example, photoreceptor degeneration, impairment of photoreceptor function, cell death, etc.

Methods for delivering a transgene (e.g., a gene encoding a CEP290 protein or a fragment thereof) to a subject are provided by the disclosure. The methods typically involve administering to a subject an effective amount of an isolated nucleic acid encoding a CEP290 protein fragment, or a rAAV comprising a nucleic acid for expressing a CEP290 protein fragment.

The human CEP290 gene consists of 52 exons, which encode for a protein of ˜290 kDa (2479 amino acids). In some embodiments, the human CEP290 gene encodes a protein comprising the amino acid sequence set forth in SEQ ID NO: 1, and as described as GenBank Accession Number (NP_079390.3). In some embodiments, the human CEP290 gene (e.g., NCBI Reference Sequence: NM_025114.3) comprises a sequence set forth in SEQ ID NO: 8.

CEP290 is a multidomain protein and contains numerous coiled-coil domains distributed over the entire length of the protein. In addition, the CEP290 protein contains membrane and microtubule-binding domains and myosin-tail homology domain. Typically, CEP290 predominantly localizes to the centrosomes and transition zone of primary cilia and to the CC of photoreceptors. Previous publications have observed that the domain of CEP290 that localizes the protein to centrosomes (e.g., the “M region” of the CEP290 gene, as described in US 2016/0185832) is necessary to mediate microtubule localization and cilium formation. In some embodiments, the “M region” refers to amino acid residues 1695 to 1966 of human CEP290, as described in US 2016/0185832.

Aspects of the instant disclosure are based, in part, on the surprising discovery that certain CEP290 fragments lacking the “M” region mediate effective rescue of cilial formation and photoreceptor rescue when expressed in a subject in need thereof, for example via administration of a viral vector (e.g., rAAV).

Accordingly in some aspects, the disclosure provides a transgene encoding a CEP290 protein fragment, wherein the CEP290 protein fragment does not comprise amino acid positions 1695 to 1966 (e.g., a region encompassing the “M” region) of SEQ ID NO: 1. A “CEP protein fragment” refers to a 2 to 2479 (e.g., any integer between 2 and 2479) amino acid portion of a CEP290 protein. In some embodiments, the CEP protein fragment comprises a contiguous amino acid portion (e.g., amino acids 580 to 1180) of CEP290 (e.g., SEQ ID NO: 1). In some embodiments, the CEP protein fragment comprises one or more (e.g., 2, 3, 4, 5, 6, 7, 8, 9, 10, or more) interrupted amino acid portions (e.g., amino acids 1 to 10, 580 to 1180 and 1967 to 2470) of CEP290 (e.g., SEQ ID NO: 1). In some embodiments, a CEP protein fragment comprises a methionine (M) amino acid residue at its N-terminus.

In some embodiments, the CEP290 protein fragment comprises at least 500 contiguous amino acids of SEQ ID NO: 1. For example, in some embodiments, the CEP290 protein fragment comprises (or consists of) amino acids 580 to 1695, or amino acids 580 to 1180, or amino acids 1181 to 1695, of CEP290 (e.g., SEQ ID NO: 1). In some embodiments, the at least 500 contiguous amino acids comprises or consists of a sequence selected from SEQ ID NOs: 2, 3 and 4.

In some embodiments, a CEP290 protein comprises amino acids 480 to 579 of CEP290 (e.g., SEQ ID NO: 1). In some embodiments, a CEP290 protein fragment comprises amino acids 480 to 580 of CEP290 (e.g., SEQ ID NO: 1). In some embodiments, a CEP290 protein comprises amino acids 480 to 880 of CEP290 (e.g., SEQ ID NO: 1). In some embodiments, a CEP290 protein comprises amino acids 580 to 880 of CEP290 (e.g., SEQ ID NO: 1). In some embodiments, a CEP290 protein comprises amino acids 580 to 1180 of CEP290 (e.g., SEQ ID NO: 1). In some embodiments, a CEP290 protein comprises amino acids 1181 to 1695 of CEP290 (e.g., SEQ ID NO: 1). In some embodiments, a CEP290 protein comprises amino acids 1181 to 1966 of CEP290 (e.g., SEQ ID NO: 1). In some embodiments, a CEP290 protein comprises amino acids 1181 to 2479 of CEP290 (e.g., SEQ ID NO: 1). In some embodiments, a CEP290 protein comprises amino acids 1696 to 1966 of CEP290 (e.g., SEQ ID NO: 1). In some embodiments, a CEP290 protein comprises amino acids 1966 to 2479 of CEP290 (e.g., SEQ ID NO: 1). In some embodiments, a CEP290 protein comprises amino acids 1800 to 2479 of CEP290 (e.g., SEQ ID NO: 1). In some embodiments, a CEP290 fragment comprises two or more (e.g., 2, 3, 4, 5, 6, or more) of the foregoing fragments. In some embodiments, a CEP290 fragment comprises two or more (e.g., 2, 3, 4, 5, 6, or more) of the foregoing fragments that are not contiguous in SEQ ID NO: 1. In some embodiments, a CEP290 protein fragment comprises or consists of the amino acid sequence set forth in any one of SEQ ID NOs: 10-19. In some embodiments, a CEP290 protein comprises amino acids 1-200 and 580-1180 of human CEP290. In some embodiments, a CEP290 protein comprises amino acids 1-200 and 580-1180 of SEQ ID NO: 1. In some embodiments, a CEP290 protein comprises the amino acid sequence set forth in SEQ ID NO: 19. In some embodiments, a CEP290 protein is encoded by the nucleic acid sequence set forth in SEQ ID NO: 29 or 34. In some embodiments, a CEP290 protein comprises amino acids 1-380 and 580-1180 of human CEP290. In some embodiments, a CEP290 protein comprises amino acids 1-380 and 580-1180 of SEQ ID NO: 1. In some embodiments, a CEP290 protein comprises the amino acid sequence set forth in SEQ ID NO: 36. In some embodiments, a CEP290 protein is encoded by the nucleic acid sequence set forth in SEQ ID NO: 35.

In some embodiments, the disclosure provides a transgene comprising a nucleic acid (e.g., isolated nucleic acid) encoding a CEP290 protein fragment. In some embodiments, the transgene comprises or consists of a nucleic acid sequence selected from SEQ ID NO: 5, 6 and 7. In some embodiments, the transgene comprises or consists of a nucleic acid sequence selected from any one of SEQ ID NOs: 20-29.

In some embodiments, the transgenes encoding a CEP290 fragment described by the disclosure mediate cilial growth and photoreceptor rescue, and are therefore useful for treating ciliopathies, for example ocular ciliopathies. Generally, a “ciliopathy” refers to a disease or disorder characterized by defective (or lack of) protein function resulting in abnormal formation or function of cilia in a cell of a subject. An “ocular ciliopathy” is a ciliopathy where abnormal formation or function of cilia occurs in ocular cells (e.g., rods, cones, photoreceptor cells, etc.) of a subject, typically resulting in retinal degeneration, loss of vision and blindness. Examples of ciliopathies include but are not limited to earlier onset developmental anomalies such as Meckel Gruber Syndrome and Joubert Syndrome, to relatively later onset diseases, such as Bardet-Biedl Syndrome, Senior-Loken Syndrome, and Usher Syndrome. In some embodiments, retinal dystrophies (e.g., due to an ocular ciliopathy) are more commonly presented in a non-syndromic manner.

In some embodiments, the ocular ciliopathy is Leber congenital amaurosis (LCA). Generally, LCA is a clinically and genetically heterogeneous disease with early onset severe retinal degeneration starting either at birth or by 5-7 years of age. In some embodiments, the LCA is LCA1, LCA2, LCA3, LCA4, LCA5, LCA6, LCA7, LCA8, LCA9, LCA10, LCA11, LCA12, LCA13, LCA14, LCA15, LCA16, or LCA17. In some embodiments, the LCA is LCA10 (e.g., LCA associated with one or more muations in a CEP290 gene). Generally, a mutation or mutations in CEP290 account for >26% of LCA (LCA10; OMIM 611755). In some embodiments, LCA is characterized by a deletion of the CEP290 gene in a subject. Generally, a mutation in CEP290 that results in LCA may be an intronic mutation, a nonsense mutation, a frameshift mutation, a missense mutation, or any combination thereof. Examples of CEP290 gene mutations associated with LCA include but are not limited to c.2991+1655A>G, c.2249T>G, c.7341dupA, c.2118_2122dupTCAGG, c.3814C>T, c.679_680delGA, c.265dupA, c.180+1G?T, c.1550delT, c.4115_4116delTA, c.4966G>T, and c.5813_5817delCTTTA, for example as described by den Hollander et al. (2006) Am J Hum Genet. 79(3):556-561. In some embodiments, the mutation in CEP290 is a deep intronic mutation, for example at position c.2991+1655A. In some embodiments, the deep intronic mutation is c.2991+1655A>G. In some embodiments, the severity of an ocular ciliopathy is modified by CEP290 mutations. For example, as described in Rao et al. (2016). Hum Mol Genet, 25(10):2005-2012. Deletions and or mutations in a CEP290 gene of a subject (e.g., a subject having or suspected of having a ciliopathy associated with a deletion or mutation of CEP290 gene) may be identified from a sample obtained from the subject (e.g., a DNA sample, RNA sample, blood sample, or other biological sample) by any method known in the art. For example, in some embodiments, a nucleic acid (e.g., DNA, RNA, or a combination thereof) is extracted from a biological samples obtained from a subject and nucleic acid sequencing is performed in order to identify a mutation in the CEP290 gene. Examples of nucleic acids sequencing techniques include but are not limited to Maxam-Gilbert sequencing, pyrosequencing, chain-termination sequencing, massively parallel signature sequencing, single-molecule sequencing, nanopore sequencing, Illumina sequencing, etc. In some embodiments, a mutation or deletion in CEP290 gene is detected indirectly, for example by quantifying CEP290 protein expression (e.g., by Western blot) or function (e.g., by analyzing cilial growth, structure, function, etc.), or by direct sequencing of the DNA and comparing the sequence obtained to a control DNA sequence (e.g., a wild-type CEP290 DNA sequence).

In some aspects, the disclosure provides a method for treating an ocular ciliopathy in a subject in need thereof, the method comprising administering to a subject having an ocular ciliopathy a therapeutically effective amount of an isolated nucleic acid, or a rAAV, as described by the disclosure. In some embodiments, the administration is subretinal administration.

An “effective amount” of a substance is an amount sufficient to produce a desired effect. In some embodiments, an effective amount of an isolated nucleic acid (e.g., an isolated nucleic acid comprising a transgene encoding a CEP290 protein fragment as described herein) is an amount sufficient to transfect (or infect in the context of rAAV mediated delivery) a sufficient number of target cells of a target tissue of a subject. In some embodiments, a target tissue is ocular tissue (e.g., photoreceptor cells, rod cells, cone cells, retinal ganglion cells, retinal cells, retinal pigmented epithelial cells, etc.). In some embodiments, an effective amount of an isolated nucleic acid (e.g., which may be delivered via an rAAV) may be an amount sufficient to have a therapeutic benefit in a subject, e.g., to increase or supplement the expression of a gene or protein of interest (e.g., CEP290), to improve in the subject one or more symptoms of disease (e.g., a symptom of an ocular ciliopathy, such as LCA), etc., such as light perception, photoreceptor function (electroretinography) and structure of the retina. The effective amount will depend on a variety of factors such as, for example, the species, age, weight, health of the subject, and the tissue to be targeted, and may thus vary among subject and tissue as described elsewhere in the disclosure.

### Isolated Nucleic Acids

In some aspects, the disclosure provides isolated nucleic acids that are useful for expressing human CEP290, or a fragment thereof. A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The skilled artisan will also realize that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

The isolated nucleic acids of the invention may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more proteins (e.g., human CEP290, or a fragment thereof). The transgene may also comprise a region encoding, for example, a miRNA binding site, and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In some embodiments, one or more additional nucleotide sequences are found between the transgene and the 5′ and/or 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV5, AAV6, AAV6.2, AAV7, AAV8, AAV9, AAV10, AAV11, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV5, AAV6, AAV6.2, AAV7, AAV8, AAV9, AAV10, AAV11, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR). Without wishing to be bound by any particular theory, a rAAV vector comprising an ITR lacking a functional TRS produces a self-complementary rAAV vector, for example as described by McCarthy (2008) Molecular Therapy 16(10):1648-1656.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences (e.g., Nrl-response element, CRX-response element, RET-1, etc.); efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., miRNA).

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931.; and Klump, H et al., Gene Therapy, 2001; 8: 811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is a CB6 promoter. In some embodiments, a transgene comprises a CB6 promoter operably linked to the nucleic acid sequence set forth in any one of SEQ ID NOs: 5-7, 20-29, and 34-35. In some embodiments, a promoter is an enhanced chicken β-actin promoter. In some embodiments, a promoter is a U6 promoter. In some embodiments, a promoter is a chicken beta-actin (CBA) promoter. In some embodiments, a transgene comprises a CBA promoter operably linked to the nucleic acid sequence set forth in any one of SEQ ID NOs: 5-7, 20-29, and 34-35. In some embodiments, a longer version of a CB promoter is used. In some embodiments, longer versions of the CB promoter enhance expression of a transgene.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the tissue-specific promoter is an eye-specific promoter. Examples of eye-specific promoters include but are not limited to a retinoschisin promoter, K12 promoter, a rhodopsin promoter, a rod-specific promoter, a cone-specific promoter, a rhodopsin kinase promoter, a GRK1 promoter, an interphotoreceptor retinoid-binding protein proximal (IRBP) promoter, retinal pigmented epithelium-specific promoter (e.g., RPE65, Best1, etc.) and an opsin promoter (e.g., a red opsin promoter, a blue opsin promoter, etc.). In some embodiments, a transgene comprises an IRBP promoter operably linked to the nucleic acid sequence set forth in any one of SEQ ID NOs: 5-7, 20-29, and 34-35.

In some embodiments, a transgene comprises a rhodopsin kinase promoter operably linked to the nucleic acid sequence set forth in SEQ ID NO: 29 or 34. In some embodiments, a transgene comprises a rhodopsin kinase promoter operably linked to the nucleic acid sequence encoding the amino acids 1-200 and 580-1180 of human CEP290. In some embodiments, a transgene comprises a rhodopsin kinase promoter operably linked to the nucleic acid sequence encoding the amino acids 1-200 and 580-1180 of SEQ ID NO: 1. In some embodiments, a transgene comprises a rhodopsin kinase promoter operably linked to the nucleic acid sequence encoding the amino acid sequence set forth in SEQ ID NO: 19.

In some embodiments, a transgene comprises a rhodopsin kinase promoter operably linked to the nucleic acid sequence set forth in SEQ ID NO: 35. In some embodiments, a transgene comprises a rhodopsin kinase promoter operably linked to the nucleic acid sequence encoding the amino acids 1-380 and 580-1180 of human CEP290. In some embodiments, a transgene comprises a rhodopsin kinase promoter operably linked to the nucleic acid sequence encoding the amino acids 1-380 and 580-1180 of SEQ ID NO: 1. In some embodiments, a transgene comprises a rhodopsin kinase promoter operably linked to the nucleic acid sequence encoding the amino acid sequence set forth in SEQ ID NO: 36.

Aspects of the disclosure relate to isolated nucleic acids comprising a transgene encoding one or more CEP290 fragments and a photoreceptor-specific promoter. A photoreceptor-specific promoter may target rod photoreceptor cells, cone photoreceptor cells, or rod and cone photoreceptor cells. In some embodiments, the photoreceptor-specific promoter is a GRK promoter (e.g., a GRK promoter). In some embodiments, a transgene comprises a GRK promoter operably linked to the nucleic acid sequence set forth in any one of SEQ ID NOs: 5-7, 20-29, and 34-35.

In some embodiments, a transgene further comprises one or more (e.g., 1, 2, 3, 4, 5, or more) introns. The length of an intron may vary. In some embodiments, an intron ranges from between about 100 nucleotides in length to about 1000 nucleotides in length (e.g., between 100 and 500, 250 and 700, 500 and 1000, etc.). In some embodiments, an intron comprises a chicken beta-actin (CBA) intron, for example as set forth in SEQ ID NO: 37. In some embodiments, an intron comprises a synthetic intron, for example an intron comprising the sequence set forth in SEQ ID NO: 38. In some embodiments, an intron comprises a MBL intron, for example as set forth in SEQ ID NO: 39. In some embodiments, an intron is positioned between a promoter (e.g., a CBA promoter, etc.) and a miniCEP290 protein coding sequence (e.g., a sequence set forth in any one of SEQ ID NOs: 5-7, 20-29, and 34-35).

In some aspects, the disclosure relates to an rAAV vector comprising an expression cassette comprising a nucleic acid encoding the sequence set forth in SEQ ID NO: 29 or 34 (e.g., a nucleic acid sequence encoding the amino acid sequence set forth in SEQ ID NO: 19) operably linked to a rhodopsin kinase (RK) promoter, wherein the expression cassette is flanked by AAV2 ITRs. In some embodiments, the rAAV vector is encapsidated by one or more AAV capsid proteins. In some embodiments, the one or more AAV capsid proteins are AAV8 capsid proteins or AAV5 capsid proteins.

In some embodiments, a promoter is a RNA polymerase III (pol III) promoter. Non-limiting examples of pol III promoters include U6 and H1 promoter sequences. In some embodiments, a promoter is a RNA polymerase II (pol II) promoter. Non-limiting examples of pol II promoters include T7, T3, SP6, RSV, and cytomegalovirus promoter sequences. In some embodiments, a pol III promoter sequence drives expression of one or more inhibitory nucleic acids and a pol II promoter sequence drives expression of a minigene.

### Recombinant Adeno-Associated Viruses (rAAVs)

In some aspects, the disclosure provides isolated AAVs. As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

In some aspects, the disclosure relates to an rAAV comprising (i) a nucleic acid (e.g., rAAV vector) comprising an expression cassette comprising a nucleic acid encoding the sequence set forth in SEQ ID NO: 29 or 34 (e.g., a nucleic acid sequence encoding the amino acid sequence set forth in SEQ ID NO: 19) operably linked to a rhodopsin kinase (RK) promoter, wherein the expression cassette is flanked by AAV2 ITRs, and (ii) one or more AAV capsid proteins. In some embodiments, the one or more capsid proteins are AAV5 or AAV8 capsid proteins.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV2, AAV3, AAV4, AAV5, AAV6, AAV7, AAV8, AAVrh8, AAV9, and AAV10. In some embodiments, an AAV capsid protein is of a serotype derived from a non-human primate, for example AAVrh8 serotype. In some embodiments, the AAV capsid protein is of a serotype that has tropism for the eye of a subject, for example an AAV (e.g., AAV5, AAV6, AAV6.2, AAV7, AAV8, AAV9, AAVrh.8, AAVrh.10, AAVrh.39 and AAVrh.43) that transduces ocular cells of a subject more efficiently than other vectors. In some embodiments, an AAV capsid protein is of an AAV8 serotype or an AAV5 serotype. In some embodiments, the AAV capsid protein comprises the sequence set forth in SEQ ID NO: 9.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the instant disclosure relates to a host cell containing a nucleic acid that comprises a coding sequence encoding a protein (e.g., a CEP290 protein fragment). In some embodiments, the instant disclosure relates to a composition comprising the host cell described above. In some embodiments, the composition comprising the host cell above further comprises a cryopreservative.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., guide RNA) from a transcribed gene.

The foregoing methods for packaging recombinant vectors in desired AAV capsids to produce the rAAVs of the disclosure are not meant to be limiting and other suitable methods will be apparent to the skilled artisan.

### Delivery of CEP290 Transgenes to the Eye

Methods for delivering a transgene to ocular (e.g., photoreceptors, such as rod cells or cone cells, retinal cells, retinal pigmented epithelial cells, etc.) tissue in a subject are provided herein. The methods typically involve administering to a subject an effective amount of an isolated nucleic acid, rAAV, or composition comprising a nucleic acid for expressing a transgene (e.g., a CEP290 protein fragment) in the subject. A subject may be any suitable mammalian organism. In some embodiments, a subject is a human. Additional examples of subjects include mouse, rat, non-human primate, pig, dog, cat, or horse subjects.

An “effective amount” of a rAAV is an amount sufficient to infect a sufficient number of cells of a target tissue in a subject. In some embodiments, a target tissue is ocular (e.g., photoreceptor, retinal, retinal pigmented epithelium, etc.) tissue. An effective amount may be an amount sufficient to have a therapeutic benefit in a subject, e.g., to improve in the subject one or more symptoms of disease, e.g., a symptom of an ocular ciliopathy (e.g., an ocular ciliopathy associated with a deletion or mutation of CEP290 gene, such as LCA). In some cases, an effective amount may be an amount sufficient to produce a stable somatic transgenic animal model. The effective amount will depend on a variety of factors such as, for example, the species, age, weight, health of the subject, and the ocular tissue to be targeted, and may thus vary among subject and tissue.

An effective amount may also depend on the rAAV used. The invention is based, in part on the recognition that rAAV comprising capsid proteins having a particular serotype (e.g., AAV5, AAV6, AAV6.2, AAV7, AAV8, AAV9, AAVrh.8, AAVrh. 10, AAVrh.39, and AAVrh.43) mediate more efficient transduction of ocular (e.g., photoreceptor, retinal, etc.) tissue that rAAV comprising capsid proteins having a different serotype. Thus in some embodiments, the rAAV comprises a capsid protein of an AAV serotype selected from the group consisting of: AAV5, AAV6, AAV6.2, AAV7, AAV8, AAV9, AAVrh.8, AAVrh.10, AAVrh.39, and AAVrh.43. In some embodiments, the rAAV comprises a capsid protein of AAV8 serotype (SEQ ID NO: 9). In some embodiments, the capsid protein comprises an amino acid sequence that is at least 70%, at least 80%, at least 90%, at least 95%, or at least 99% identical to SEQ ID NO: 9. In some embodiments, the capsid protein is AAV5 capsid protein.

In certain embodiments, the effective amount of rAAV is 10¹⁰, 10¹¹, 10¹², 10¹³, or 10¹⁴ genome copies per kg. In certain embodiments, the effective amount of rAAV is 10¹⁰, 10¹¹, 10¹², 10¹³, 10¹⁴, or 10¹⁵ genome copies per subject.

An effective amount may also depend on the mode of administration. For example, targeting an ocular (e.g., photoreceptor, retinal, etc.) tissue by intrastromal administration or subcutaneous injection may require different (e.g., higher or lower) doses, in some cases, than targeting an ocular (e.g., photoreceptor, retinal, etc.) tissue by another method (e.g., systemic administration, topical administration, subretinal administration, etc.). In some embodiments, intrastromal injection (IS) of rAAV having certain serotypes (e.g., AAV5, AAV6, AAV6.2, AAV7, AAV8, AAV9, AAVrh.8, AAVrh.10, AAVrh.39, and AAVrh.43) mediates efficient transduction of ocular (e.g., corneal, photoreceptor, retinal, etc.) cells. Thus, in some embodiments, the injection is intrastromal injection (IS). In some embodiments, the administration is via injection, optionally subretinal injection or intravitreal injection. In some embodiments, the injection is subretinal injection. In some embodiments, the injection is superchoroidal injection. In some embodiments, the injection is topical administration (e.g., topical administration to an eye). In some cases, multiple doses of a rAAV are administered.

Without wishing to be bound by any particular theory, efficient transduction of ocular (e.g., photoreceptor, retinal, retinal pigmented epithelial, etc.) cells by rAAV described herein may be useful for the treatment of a subject having an ocular disease (e.g., an ocular ciliopathy). Accordingly, methods and compositions for treating ocular disease are also provided herein. In some aspects, the disclosure provides a method for treating an ocular ciliopathy (e.g., an ocular ciliopathy associated with a deletion or mutation of CEP290 gene), the method comprising: administering to a subject having or suspected of having an ocular ciliopathy an effective amount of rAAV, wherein the rAAV comprises (i) a capsid protein having a serotype selected from the group consisting of AAV5, AAV6, AAV6.2, AAV7, AAV8, AAV9, AAVrh.8, AAVrh.10, AAVrh.39, and AAVrh.43, and (ii) a nucleic acid comprising a promoter operably linked to a transgene (e.g., a transgene encoding a CEP290 protein fragment as described by the disclosure).

In some embodiments, administration of a rAAV (or isolated nucleic acid) as described by the disclosure results in transduction of a cell or cells comprising a cilium, optionally a photoreceptor sensory cilium. The photoreceptor (PR) sensory cilium is nucleated from the basal body at the apical surface of the inner segment. As the microtubules extend, they form a doublet microtubule structure, called the connecting cilium (CC). The CC is analogous to the transition zone of a prototypic cilium and extends into the outer segment (OS) of the photoreceptor cell. The CC is acts as a conduit for unidirectional or bidirectional transport of cargo moieties between the inner and the outer segments. The CC also acts as a ‘gatekeeper’ to regulate the entry or exit of the cargo, which aids in the maintenance of its unique composition. In some embodiments, administration of a rAAV (or isolated nucleic acid) as described by the disclosure results in growth or formation of a photoreceptor sensory cilium, a connecting cilium, or a combination thereof.

In some embodiments, delivery of a rAAV (or isolated nucleic acid) as described by the disclosure, for example miniCEP290-1-200/580-1180, results in improved structural and/or functional rescue (e.g., as measured by ERG, immunofluorescence analysis, etc.) relative to previously described miniCEP290 vectors (e.g., miniCEP290-580-1180). In some embodiments, delivery of the rAAV improves structural and/or functional rescue by between 2-fold and 100-fold (e.g., 2, 3, 4, 5, 10, 20, 25, 50, 75, 100-fold). In some embodiments, delivery of the rAAV improves structural and/or functional rescue of more than 100-fold (e.g., 100-fold, 200-fold, 300-fold, 400-fold, 500-fold, etc.).

The rAAVs may be delivered to a subject in compositions according to any appropriate methods known in the art. The rAAV, preferably suspended in a physiologically compatible carrier (i.e., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments, a host animal does not include a human.

Delivery of the rAAVs to a mammalian subject may be by, for example, intraocular injection, subretinal injection, superchoroidal injection, or topical administration (e.g., eye drops). In some embodiments, the intraocular injection is intrastromal injection, subconjunctival injection, or intravitreal injection. In some embodiments, the injection is not topical administration. Combinations of administration methods (e.g., topical administration and intrastromal injection) can also be used.

The compositions of the disclosure may comprise an rAAV alone, or in combination with one or more other viruses (e.g., a second rAAV encoding having one or more different transgenes, such as a plurality of rAAVs where each rAAV encodes a different CEP290 fragment). In some embodiments, a composition comprises 1, 2, 3, 4, 5, 6, 7, 8, 9, 10, or more different rAAVs each having one or more different transgenes. The skilled artisan recognizes that in some embodiments, a subject is administered a plurality of isolated nucleic acids (or vectors, such as plasmids, lentiviral vectors, etc.), where each nucleic acid of the plurality encodes a different CEP290 fragment.

In some embodiments, a composition further comprises a pharmaceutically acceptable carrier. Suitable carriers may be readily selected by one of skill in the art in view of the indication for which the rAAV or composition is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions (e.g., compositions comprising one or more rAAVs) are administered in sufficient amounts to transfect the cells of a desired tissue (e.g., ocular tissue, such as photoreceptor, retinal, etc., tissue) and to provide sufficient levels of gene transfer and expression without undue adverse effects. Examples of pharmaceutically acceptable routes of administration include, but are not limited to, direct delivery to the selected organ (e.g., subretinal delivery to the eye), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors.

An effective amount of an rAAV is an amount sufficient to target infect an animal, target a desired tissue. The effective amount will depend primarily on factors such as the species, age, weight, health of the subject, and the tissue to be targeted, and may thus vary among animal and tissue. In some embodiments, the volume of an rAAV (e.g., a composition comprising an rAAV) administered ranges from about 10 μl to about 1000 μl per eye. In some embodiments, an rAAV is administered at a dosage between about 10¹¹ to about 10¹³ genome copies per eye, or from about 10¹¹ to about 10¹⁴ rAAV genome copies/ml. In some embodiments, an effective amount is produced by multiple doses of an rAAV.

In some embodiments, a dose of rAAV is administered to a subject no more than once per calendar day (e.g., a 24-hour period). In some embodiments, the dose is administered to a subject only once in the lifetime of the subject. In some embodiments, a single dose is administered in each eye of the subject only once in the lifetime of the subject. In some embodiments, administration is bilateral administration, and the doses may be administered about 1 week, about 2 weeks, about 3 weeks, about 4 weeks, about 1 month, or about 2 months apart. In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year). In some embodiments, a dose of rAAV is administered postnatally. In some embodiments, a dose of rAAV is administered postnatally between day 7 and day 13. In some embodiments, a dose of rAAV is administered postnatal day 10.

In some embodiments, rAAV compositions are formulated to reduce aggregation of AAV particles in the composition, particularly where high rAAV concentrations are present (e.g., ˜10¹³ GC/ml or more). Appropriate methods for reducing aggregation of may be used, including, for example, addition of surfactants, pH adjustment, salt concentration adjustment, etc. (See, e.g., Wright FR, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, rAAVs in suitably formulated pharmaceutical compositions disclosed herein are delivered directly to target tissue, e.g., direct to ocular tissue (e.g., photoreceptor, retinal, etc., tissue) However, in certain circumstances it may be desirable to separately or in addition deliver the rAAV-based therapeutic constructs via another route, e.g., subcutaneously, intrapancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by intravitreal injection or subretinal injection.

The pharmaceutical forms suitable for injectable use include suspension-based formulations, sterile aqueous solutions or dispersions, and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a suitable sterile aqueous medium may be employed. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μ) should be designed using polymers able to be degraded in vivo. Biodegradable polyalkyl-cyanoacrylate nanoparticles that meet these requirements are contemplated for use.

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, the instant disclosure relates to a kit for producing a rAAV, the kit comprising a container housing an isolated nucleic acid comprising a transgene encoding a CEP290 protein fragment having the amino acid sequence set forth in any one of SEQ ID NOs: 2-4 and 10-19. In some embodiments, the kit further comprises a container housing an isolated nucleic acid encoding an AAV capsid protein, for example an AAV8 capsid protein (e.g., SEQ ID NO: 9) or an AAV5 capsid protein.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

Exemplary embodiments of the invention will be described in more detail by the following examples. These embodiments are exemplary of the invention, which one skilled in the art will recognize is not limited to the exemplary embodiments.

### EXAMPLES

### Example 1

### Therapeutic Strategies for CEP290-LCA

The relative sparing of the central region of the CEP290-LCA patient retinas indicates that gene therapy may be a viable option for visual restoration in patients. However, progress in the development of mutation-independent gene replacement strategies for CEP290-LCA has been delayed largely because of unsuitability of the long CEP290 gene to be packaged into conventional AAV vector system for gene therapy. This example describes delivery of CEP290 fragments via AAV to treat CEP290-LCA. In some embodiments, the described CEP290 fragments restore cilial growth and photoreceptor function in a mutation-independent manner, and are thus useful for treatment of nonsyndromic LCA and retinal degeneration in systemic ciliopathies due to CEP290 mutations.

The full-length CEP290 cDNA is ˜8 kb long, which generally exceeds the packaging limit of conventional AAV vectors. A schematic depiction of the full-length CEP290 gene representing the locations of distinct protein interaction domains is shown in FIG. 1. Here, CEP290 fragments that retain function in photoreceptors (PR) and can be delivered using the conventional AAV vectors were identified. As CEP290 is a ciliary protein and regulates cilia growth, an in vitro assay of cilia growth was developed in order to use as a surrogate marker to test the function of shorter CEP290 regions. It was observed that mouse embryonic fibroblasts (MEFs) derived from a Cep290-mutant (Cep290^{rd16}) mouse, which recapitulates the early onset severe PR degeneration phenotype, have fewer ciliated cells and the cells that formed cilia were shorter compared to controls. This observation is consistent with previous studies that revealed fewer and shorter cilia in fibroblasts derived from CEP290-LCA patient samples.

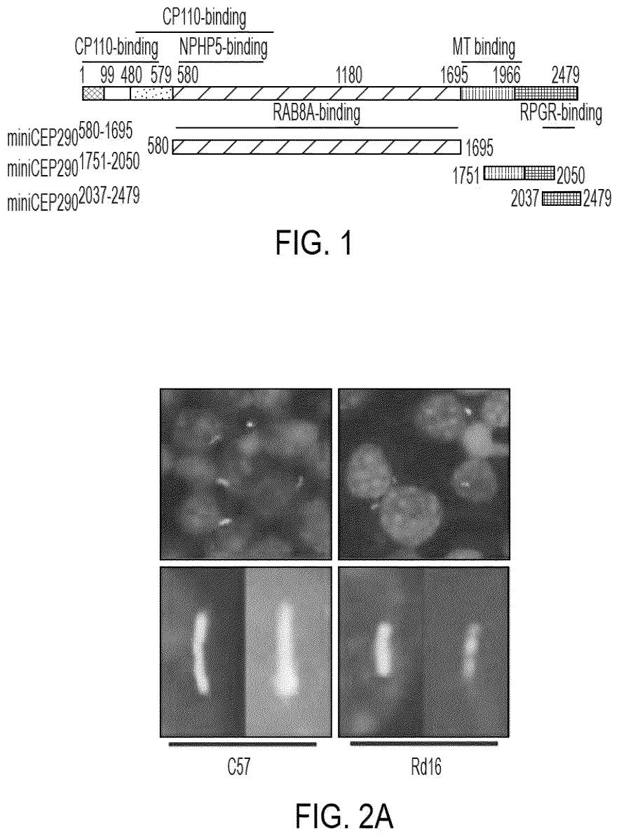

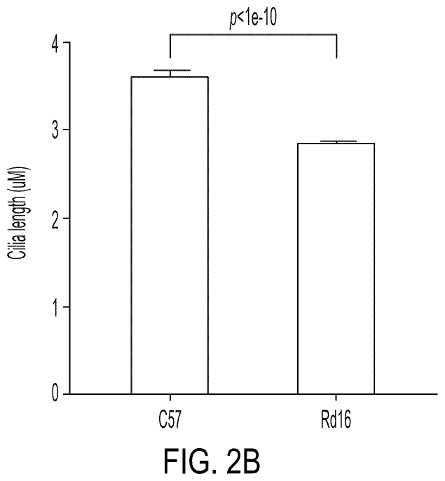

As shown in FIGS. 2A-2B, cilia of Cep290^{rd16} MEFs are ˜2.7 μm in length as compared to controls, which have ˜3.8 μm long cilia. In addition, fewer cells with cilia were detected among Cep290^{rd16} MEFs as compared to controls.

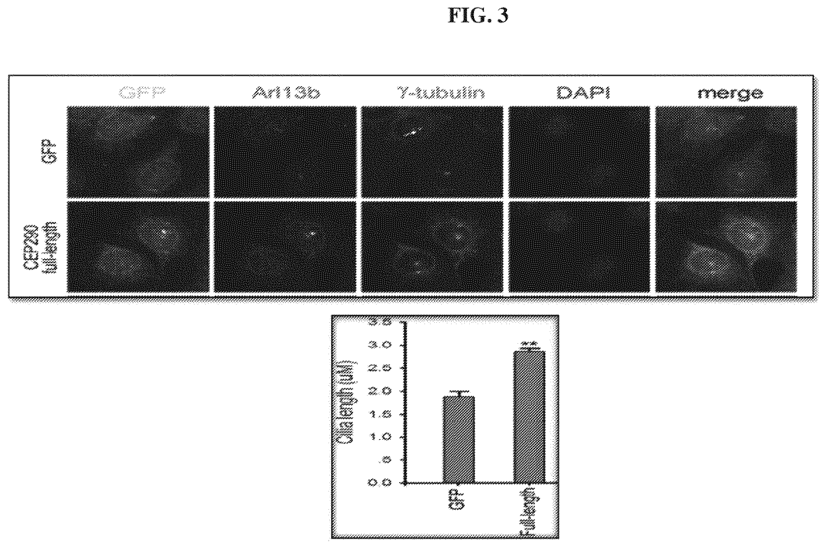

Next, the effect of expressing full-length human CEP290 protein on cilia length in Cep290^{rd16} MEFs was investigated. It was observed that the full-length human CEP290 protein correctly localizes to cilia, as determined by co-staining with ARL13b, which is a cilia marker (FIG. 3). Expressing GFP protein did not result in its localization to cilia. Additionally, measurement of cilia length showed that expression of CEP290 protein significantly rescued the cilia length of Cep290^{rd16} MEFs as compared to expression of GFP.

Construction of vCEP290

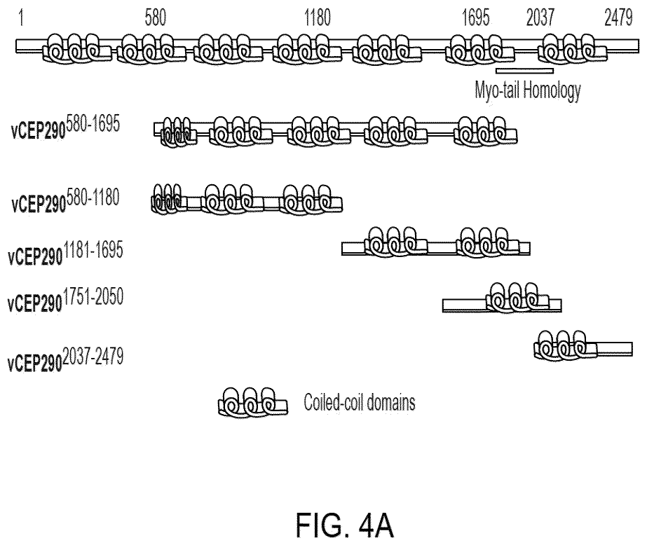

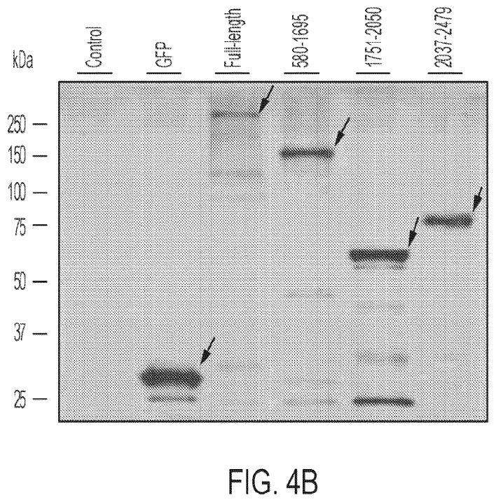

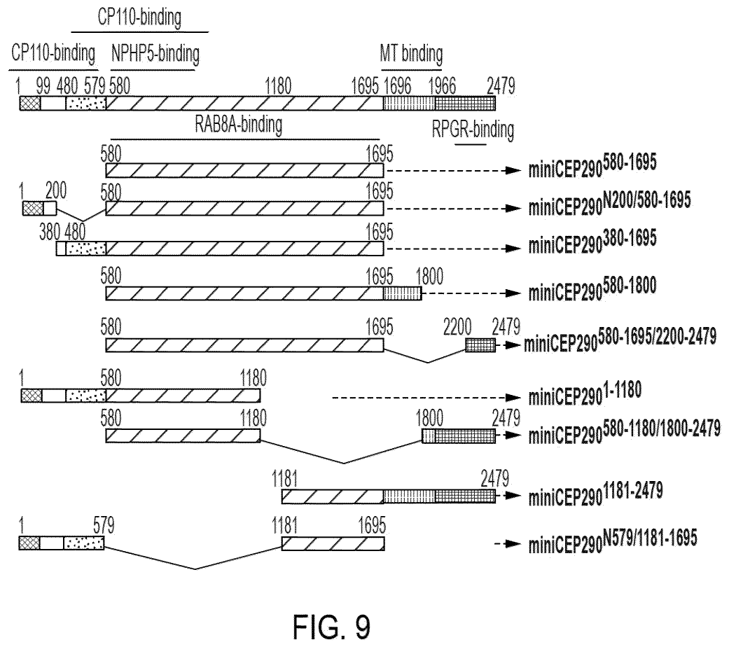

The CEP290 gene encodes a predominantly coiled-coil protein. Constructs that removed repetitive domains of human CEP290, such as plasmids encoding GFP-fused miniCEP290⁵⁸⁰⁻¹⁶⁹⁵, miniCEP290¹⁷⁵¹⁻²⁰⁵⁰ and miniCEP290²⁰³⁷⁻²⁴⁷⁹ (FIG. 4A), were produced. Variants were cloned into pEGFP-C1 vector expressing the gene under the control of CMV promoter. The constructs express stable CEP290 protein fragments as determined by immunoblot analysis of protein extracts from transiently transfected mouse embryonic fibroblasts (FIG. 4B; see arrows). To test the functional potential of the miniCEP290s, a surrogate assay system using Cep290^{rd16} MEFs (mouse embryonic fibroblasts) was used. FIG. 9 shows additional examples of CEP290 variants.

Effect of vCEP290 on Cilia Length

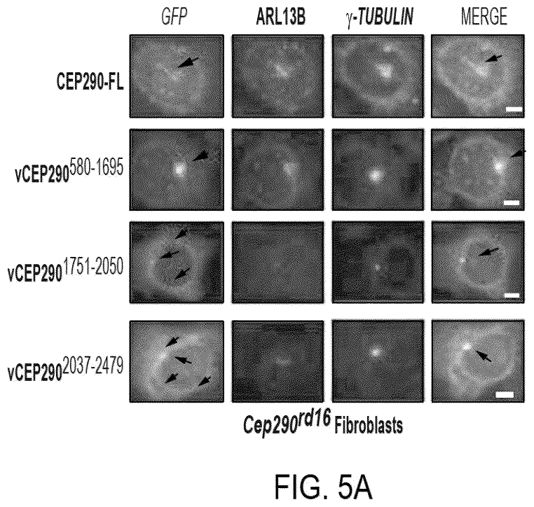

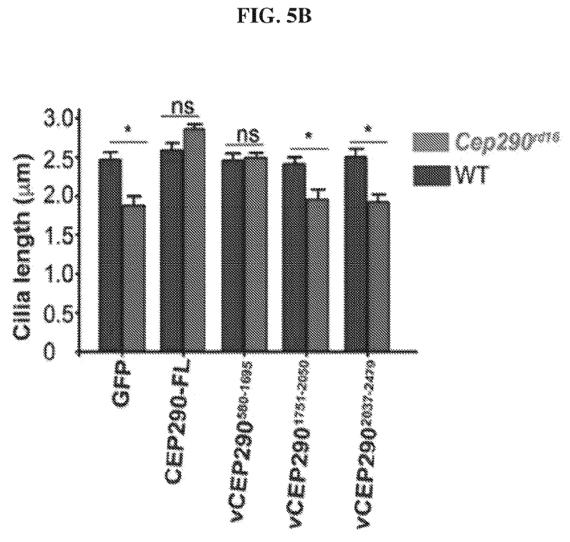

As shown in FIG. 5A, expression of different GFP-vCEP290-encoding plasmids into Cep290^{rd16} or wild type mouse embryonic fibroblasts indicates that vCEP290⁵⁸⁰⁻¹⁶⁹⁵ localizes predominantly to the basal bodies (co-localization with γ-tubulin) and proximal cilia (co-localization with ADP-Ribosylation Factor-Like 13B; ARL13B; ciliary marker). Expression of other variants indicated a relatively diffuse pattern of localization. The ability of the vCEP290 to modulate cilia length in Cep290^{rd16} fibroblasts was then assessed. As shown in FIG. 5B, cilia length of the mutant fibroblasts was significantly increased when vCEP290⁵⁸⁰⁻¹⁶⁹⁵ was expressed. Other variants, and the negative control expressing only GFP, did not reveal a change in the cilia length of the fibroblasts. No effect on cilia length of the wild type fibroblasts was observed.

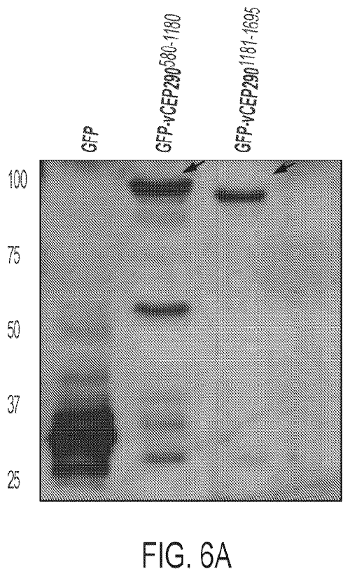

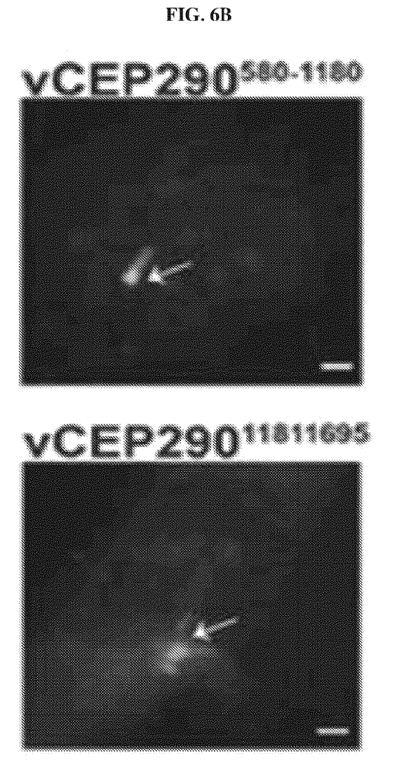

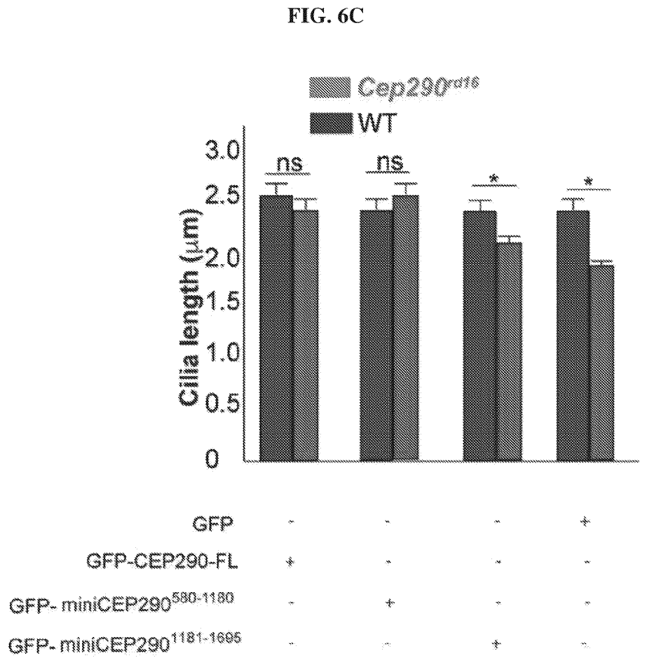

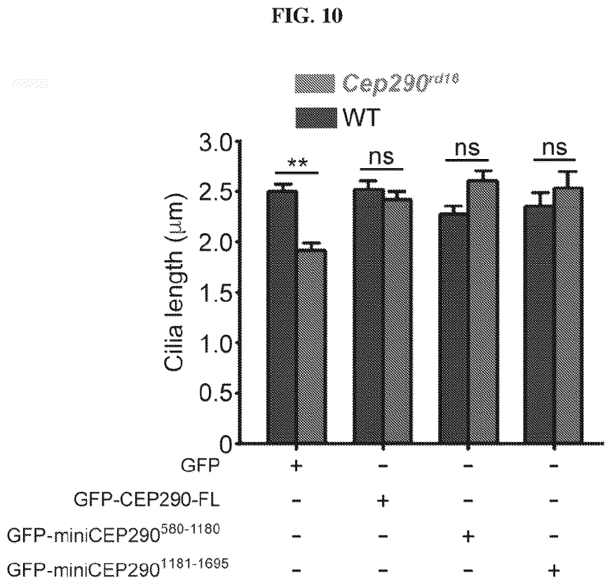

Whether further shortening vCEP290⁵⁸⁰⁻¹⁶⁹⁵ will result in a cilia length rescue was then investigated. Plasmids encoding GFP-fused vCEP290⁵⁸⁰⁻¹¹⁸⁰ and vCEP290¹¹⁸¹⁻¹⁶⁹⁵ were produced and their expression, localization and potential to rescue cilia length in Cep290^{rd16} fibroblasts were tested. Both variants exhibited optimal expression as determined by immunoblotting using anti-GFP antibody, and localization to cilia (FIGS. 6A-6B). Data for vCEP290¹¹⁸¹⁻¹⁶⁹⁵ indicate predominant localization to the base of cilia and diffuse staining around the basal body. Cilia rescue assay data indicate that expression of either variant results in a significant increase in the cilia length of Cep290^{rd16} fibroblasts (FIG. 6C and FIG. 10).

Potential of vCEP290 in Vivo

Functionality of vCEP290 constructs in vivo was investigated. vCEP290⁵⁸⁰⁻¹¹⁸⁰, vCEP290¹¹⁸¹⁻¹⁶⁹⁵ and vCEP290²⁰³⁷⁻²⁴⁷⁹ (as negative control since it did not rescue the cilia length defect in the fibroblasts) were cloned into an AAV2 vector having a CBA promoter and containing an IRES (internal ribosome entry site) between the gene of interest (e.g., vCEP290) and GFP. This permits both CEP290 and GFP to be translated from a single bicistronic mRNA and assists in identifying transduced photoreceptors using an anti-GFP antibody. Each rAAV (e.g., AAV2/8-CBA-vCep290⁵⁸⁰⁻¹¹⁸⁰-IRES-GFP, AAV2/8-CBA-vCep290¹¹⁸¹⁻¹⁶⁹⁵-IRES-GFP, AAV2/8-CBA-vCep290²⁰³⁷⁻²⁴⁷²-IRES-GFP, and negative control AAV2/8-CBA-GFP) were injected at 8×10⁹ vg/eye in 1 μl volume into the subretinal space of Cep290^{rd16} pups at P0 stage. The mice were assessed for PR function and retinal morphology up to 5 weeks after injection.

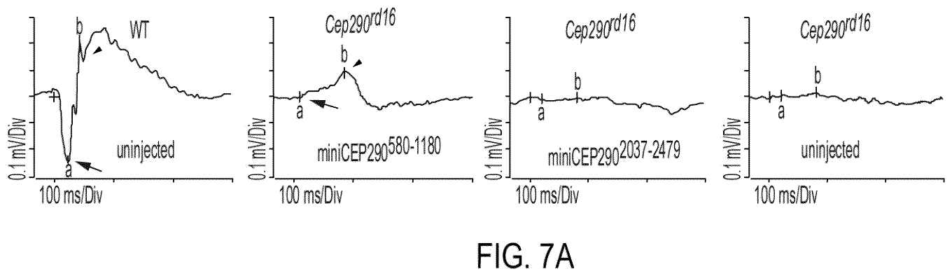

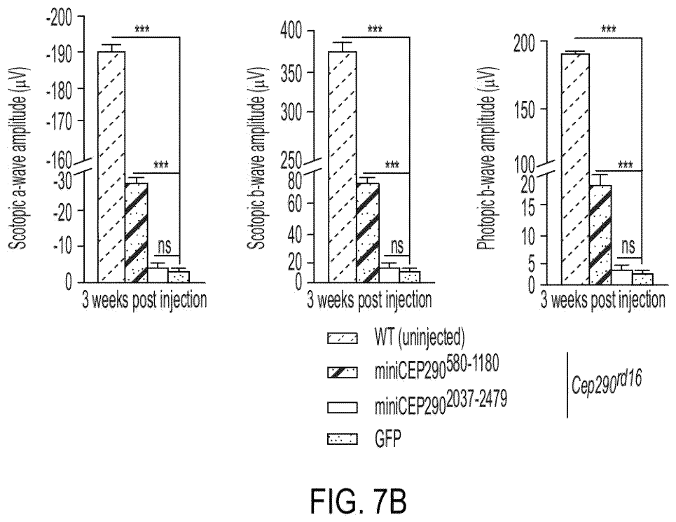

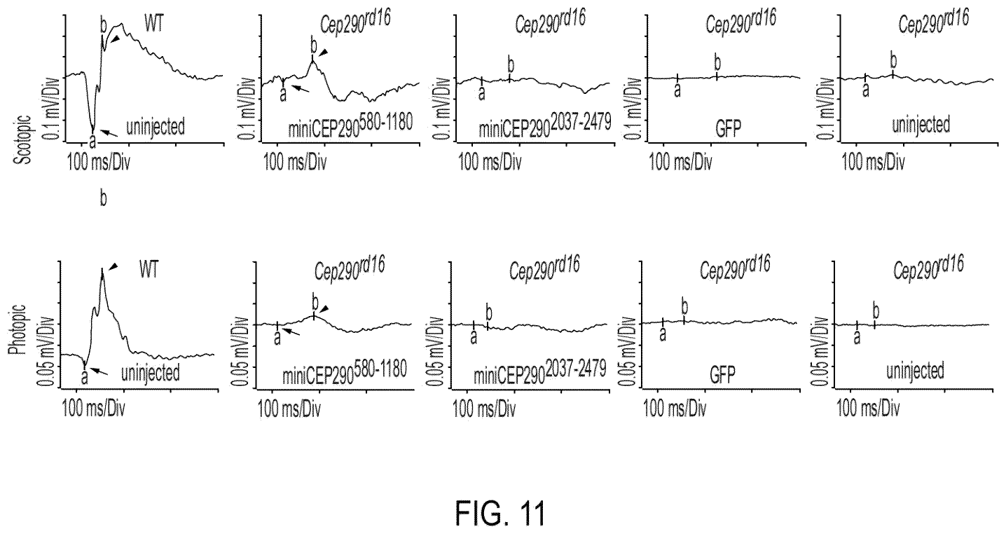

Analysis of PR function by electroretinography (ERG) at 3 weeks post-injection revealed improvement (25-30%) in both scotopic (rod PR-mediated) and photopic (cone PR-mediated) (FIGS. 7A-7B, and FIGS. 11-12) responses of the miniCEP290⁵⁸⁰⁻¹¹⁸⁰-injected mice. No improvement was detected using miniCEP290²⁰³⁷⁻²⁴⁷⁹ or GFP. Further analysis revealed that the improvement in the ERG was stable up to 4 weeks post injection.

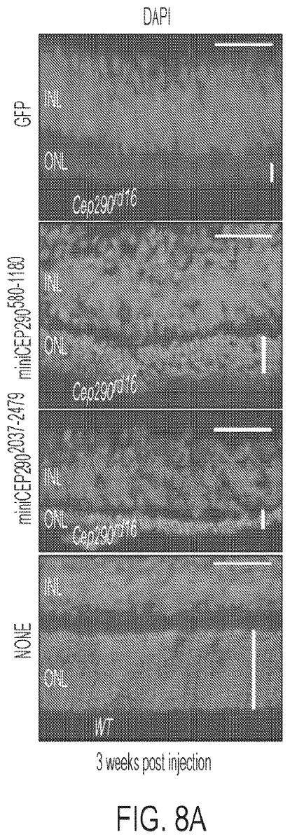

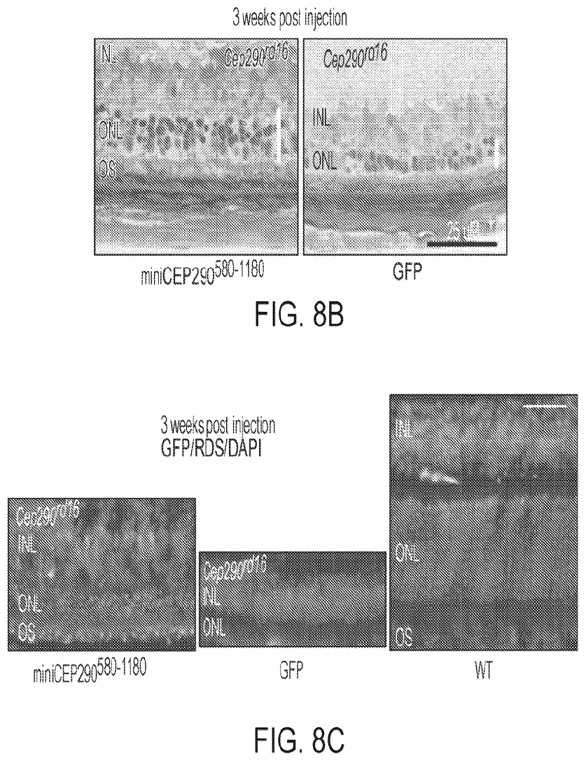

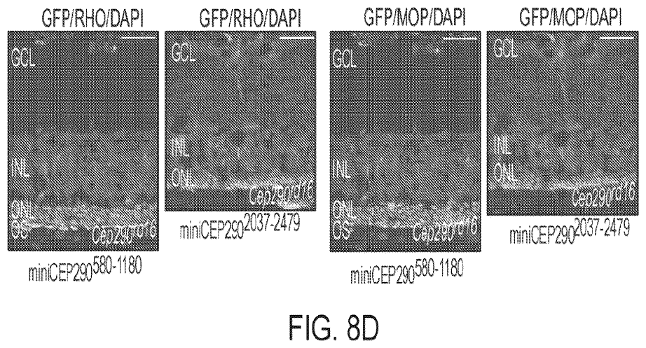

The number of layers of the ONL, which correlates with PR survival, were also counted in retinal cryosections: ˜6-7 layers were observed in Cep290^{rd16} retinas injected with miniCep290⁵⁸⁰⁻¹¹⁸⁰; 4-5 layers were observed in Cep290^{rd16} retinas injected with miniCep290¹¹⁸¹⁻¹⁶⁹⁵ and; 2-3 layers were observed in retinas injected with miniCep290²⁰³⁷⁻²⁴⁷² or GFP (equivalent to uninjected Cep290^{rd16} at 3 weeks of age), as shown in FIG. 8A. It was also observed that ultrathin sections of the CEP290^{rd16} retinas injected with miniCEP290⁵⁸⁰⁻¹¹⁸⁰ exhibited significant preservation of the outer nuclear layer (ONL) (FIG. 8B).

The structural preservation of photoreceptor (PR) outer segment in the miniCEP290⁵⁸⁰⁻¹¹⁸⁰-injected mice was examined by staining with peripherin-RDS (retinal degeneration slow, PR outer segment marker 45). RDS is a structural protein that specifically localizes to the outer segment (OS) discs and maintains the OS structure. The miniCEP290⁵⁸⁰⁻¹¹⁸⁰-injected Cep290^{rd16} mice exhibited improved RDS localization to the outer segment as compared to undetectable RDS expression in the GFP-injected mice (FIG. 8C). The expression of rhodopsin and cone opsins, two of the key phototransduction proteins, was also examined. Undetectable opsin expression was detected in the miniCEP290²⁰³⁷⁻²⁴⁷⁹-injected retinas. However, the miniCEP290⁵⁸⁰⁻¹¹⁸⁰-injected retinas revealed detectable expression of rhodopsin and cone opsins in the outer segments (FIG. 8D). Some staining of cone opsins in the inner segment and outer nuclear layer was also observed. Overall, the data indicate that the expression of miniCEP290⁵⁸⁰⁻¹¹⁸⁰ can improve the function, morphology and opsin trafficking of CEP290^{rd16} retinas.

### Materials and Methods

### Cell Culture, Transient Transfection and Immunostaining

MEFs derived from the WT and Cep290^{rd16} mice were maintained in DMEM with 10% FBS. Transient transfection with GFP-CEP290-FL or GFP-miniCEP290s was performed using Lipofectamine 2000 (Thermo Fisher). The transfected cells were either harvested for immunoblotting or were serum-starved to induce cilia growth. The ciliated cells were then immunostained, imaged under Leica microscope (DM5500). Images were then processed for cilia length evaluation using Image J.

### Constructs and AAV Production

For in vitro experiments, full-length or miniCEP290-expressing cDNAs were cloned into pEGFP-C1 plasmid expressing GFP-tagged proteins under the control of CMV promoter. For AAV production, the miniCEP290-encoding cDNAs were cloned into a pAAV2 vector plasmid between a CMVenhancer/CBA (chicken β-actin) promoter upstream of IRES (internal ribosome entry site) GFP and β-globin intron. This expression cassette was flanked with AAV2 inverted terminal repeats (ITRs). The recombinant AAV2 genomes were packaged with AAV8 capsid by HEK293-triple transfection method and purified by CsCl gradient centrifugation method.

### Subretinal Injection

Wild type C57BL6/J mice were obtained from a commercial source. The Cep290^{rd16} mice were also obtained. The Cep290^{rd16} mouse pups (P0/P1) were subretinally injected unilaterally with 8×10⁹ vg/μl (total volume 1 μl) of the virus.

### ERG and Immunofluorescence Microscopy of the Retina

Scotopic and photopic ERGs were performed. For scotopic response, mice were dark adapted overnight and all procedures were performed under dim red light. Light adapted (photopic) ERGs were recorded after light adaptation with a background illumination of 30 cd/m² (white 6500 K) for 8 min.

Immunofluorescence microscopy was performed by staining retinal cryosection sections with primary antibodies: rhodopsin, M-opsin, and peripherin-RDS, ARL13B, GFP (Abcam), and γ-tubulin. After washing with PBS (phosphate buffered saline), Alexa-488 or Alexa-546-conjugated secondary antibodies were added and the sections were further incubated for 1 h. After washing, nuclei were stained with DAPI and cells were imaged using a Leica microscope (DM5500).

### Example 2

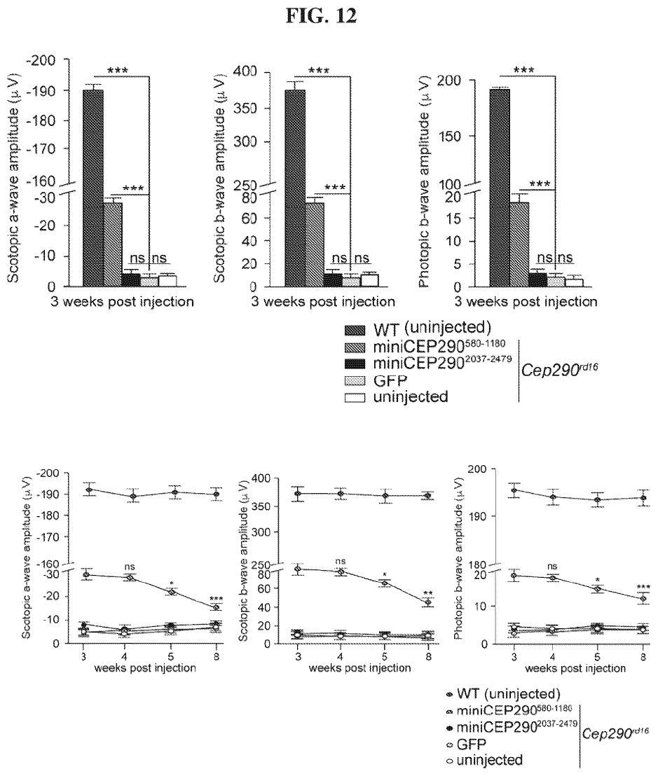

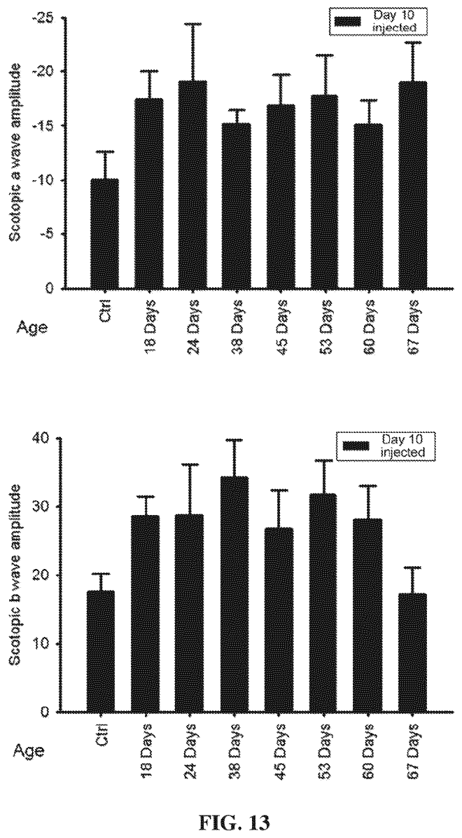

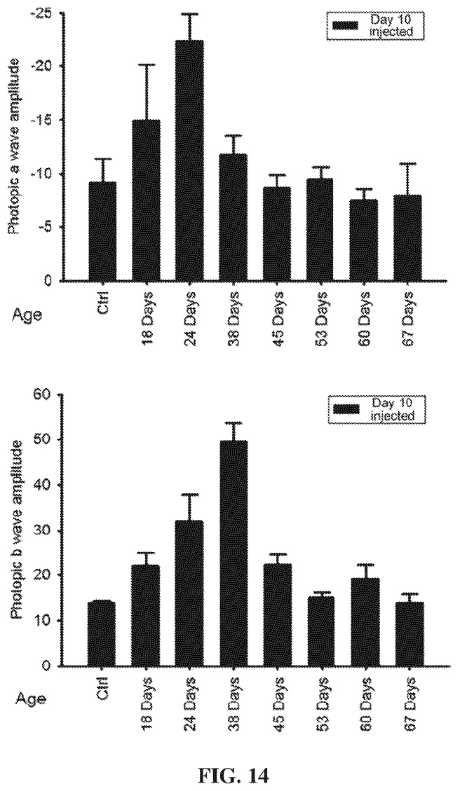

This example describes in vivo experiments to investigate CEP290 minigene expression. Briefly, 10-day old rd16 mice were subretinally injected with a CEP290 minigene construct. Expression of the CEP290 minigene was driven by a GRK promoter. Post-injection, rod (scotopic) and cone (photopic) photoreceptor response was assessed. FIG. 13 shows scotopic a-wave and b-wave amplitude for 10-day old mice subretinally injected with the indicated miniCEP290 construct (GRK-580-1180). ERG were recorded at the indicated days after injection. FIG. 14 shows photopic a-wave and b-wave amplitude for 10-day old mice subretinally injected with the indicated miniCEP290 construct (GRK-580-1180). ERG were recorded at the indicated days after injection. Data indicate a sustained response in rod cells of up to 67 days, and an increase in cone cell activity, relative to miniCEP290 constructs driven by a CB6 promoter.

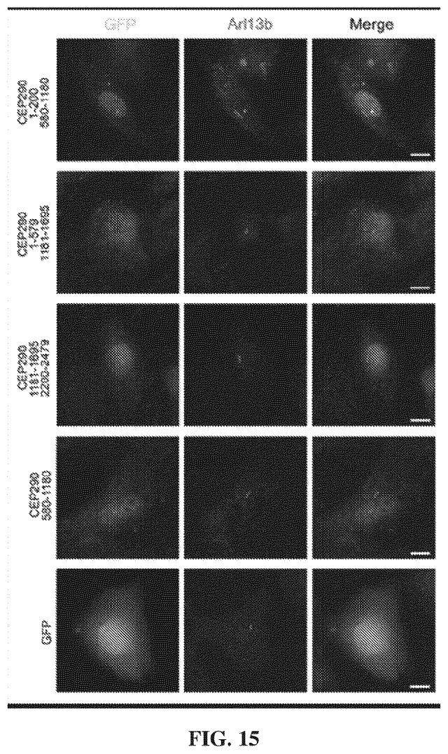

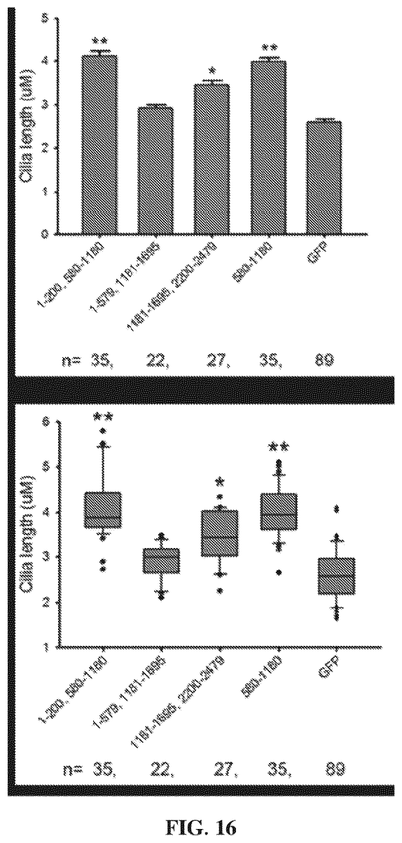

In vitro experiments were also performed. FIG. 15 shows micrographs of rd16 mouse embryonic fibroblasts transiently transfected with cDNA encoding the indicated minigenes encoding the protein and GFP. Expression of the minigenes was driven by a CB6 promoter. FIG. 16 shows data for cilia length measurement of rd16 mouse embryonic fibroblasts transiently transfected with cDNA encoding the indicated minigenes encoding the protein and GFP. Data indicate an increase in cilia length in miniCEP290 transfected cells relative to control (GFP) transfected cells.

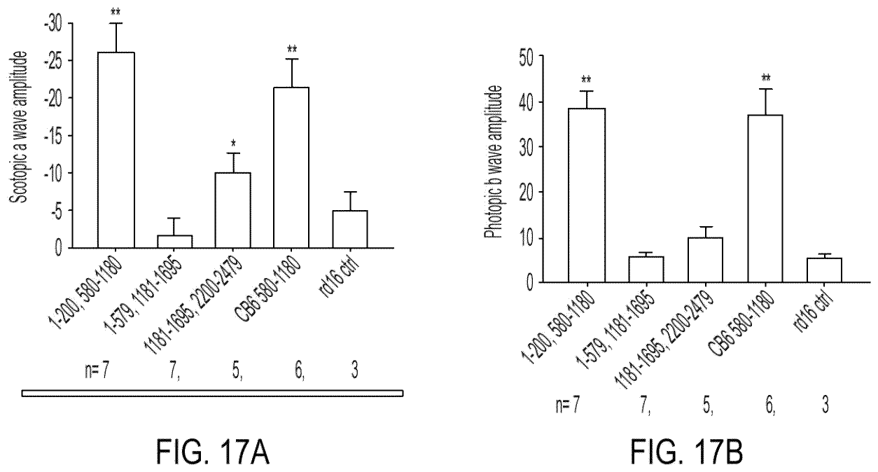

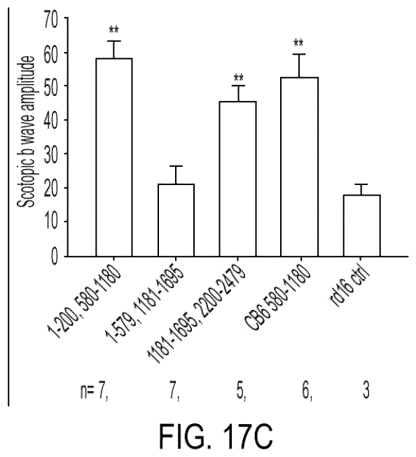

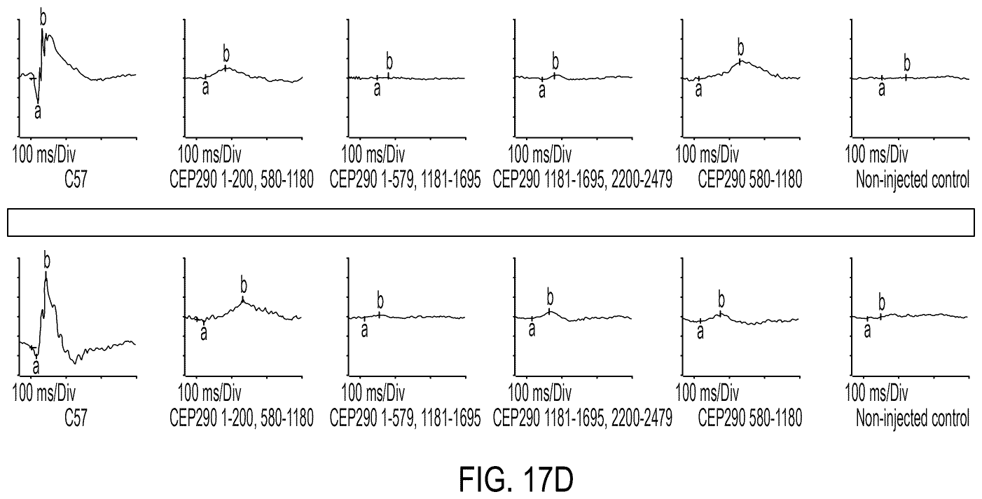

FIGS. 17A-17D show scotopic and photopic wave amplitudes of CB6-promoter driving the indicated minigenes were subretinally injected into 10 day old rd16 mice. FIG. 17A shows scotopic a-wave amplitude. FIG. 17B shows photopic b-wave amplitude. FIG. 17C shows scotopic b-wave amplitude. FIG. 17D shows ERG of rd16 mice injected with CB6-1-200-580-1180 miniCEP290. ERG were recorded at indicated days after injection and compared to others.

### Example 3

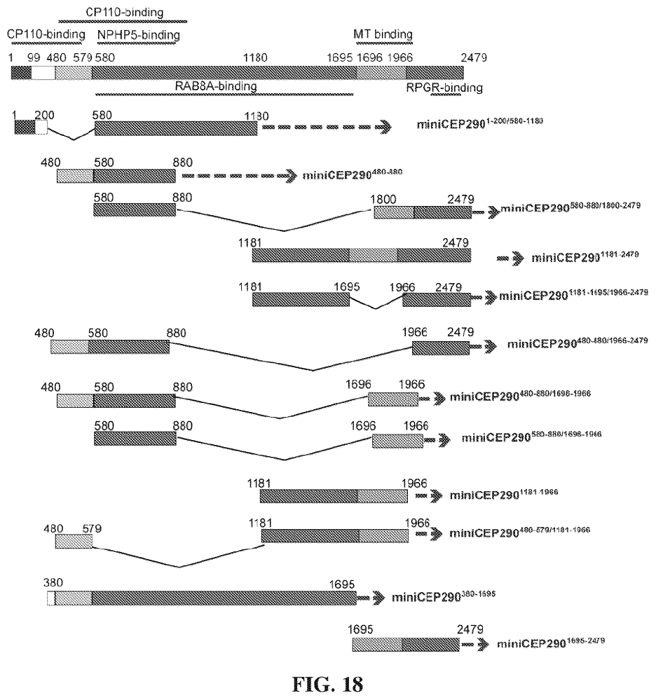

FIG. 18 is a schematic depicting additional embodiments of CEP290 minigenes. In some embodiments, expression of the minigenes is driven by a CB6 promoter. In some embodiments, expression of the minigenes is driven by an eye-specific promoter, for example a Rhodopsin Kinase (RK) promoter such as a GRK promoter.

Mice at P10 stage were subretinally injected with the AAV-vectors encoding the minigene constructs. The minigene was encapsulated in an AAV8 capsid protein. The ERG was performed at the indicated ages after injection, as described in the Figures.

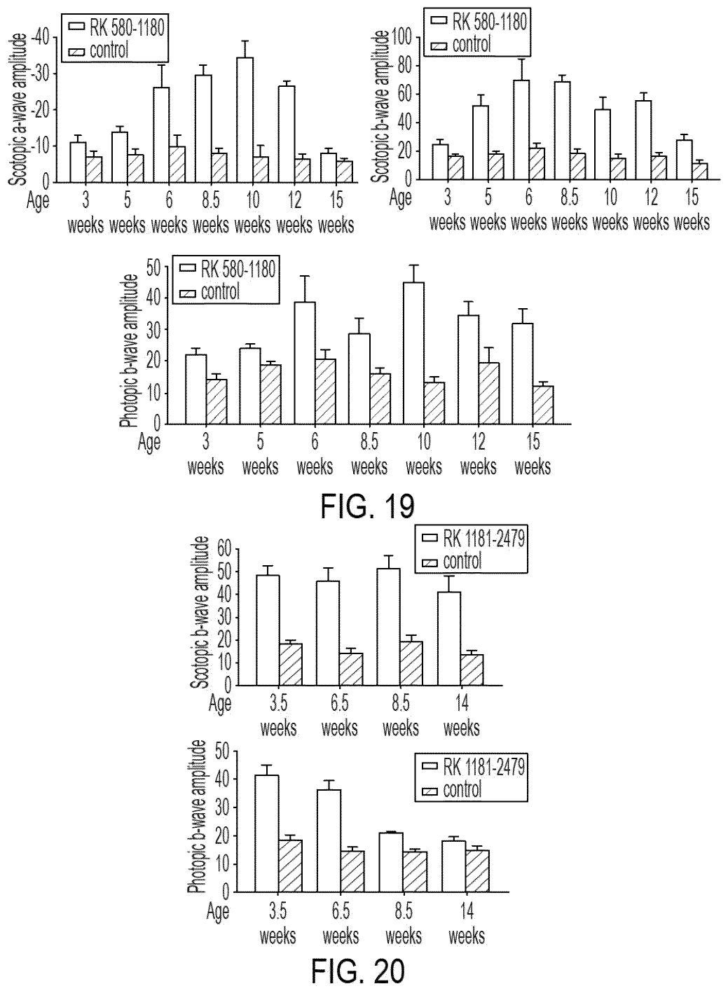

FIG. 19 shows representative data for ERG analysis of Rhodopsin Kinase (RK) promoter-driven expression of miniCEP290-580-1180 in subretinally-injected mice. Rescue effect lasted more than 10 weeks post-injection.

FIG. 20 shows representative data for ERG analysis of Rhodopsin Kinase (RK) promoter-driven expression of miniCEP290-1181-2479 in subretinally-injected mice. Rescue effect lasted up to 8.5 weeks post-injection.

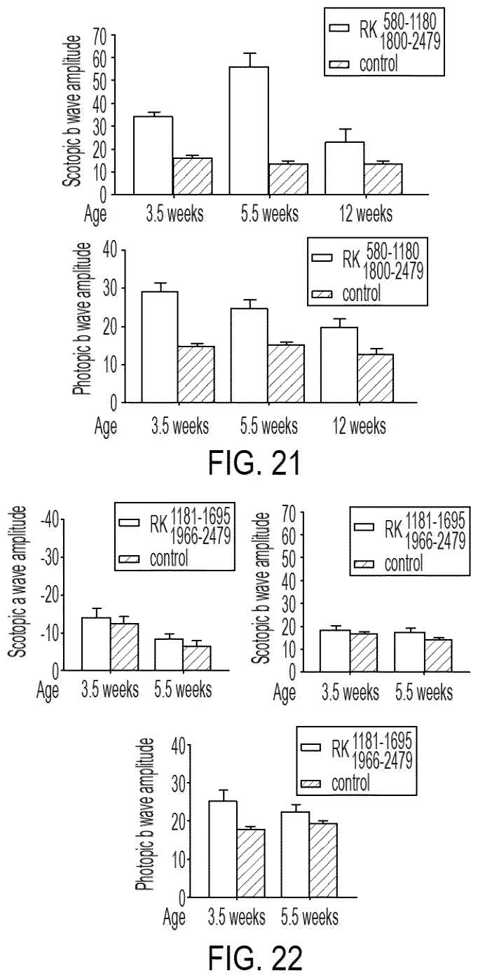

FIG. 21 shows representative data for ERG analysis of Rhodopsin Kinase (RK) promoter-driven expression of miniCEP290-580-1180/1800-2479 in subretinally-injected mice. Rescue effect lasted up to 5.5 weeks post-injection.

FIG. 22 shows representative data for ERG analysis of Rhodopsin Kinase (RK) promoter-driven expression of miniCEP290-1181-1695/1966-2479 in subretinally-injected mice.

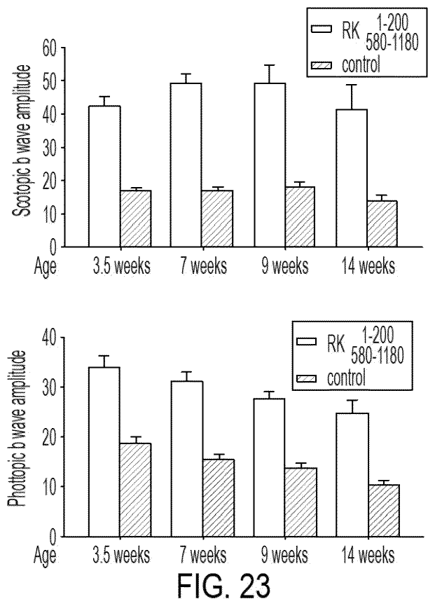

FIG. 23 shows representative data for ERG analysis of Rhodopsin Kinase (RK) promoter-driven expression of miniCEP290-1-200/580-1180 in subretinally-injected mice. Rescue effect lasted more than 14 weeks post-injection.

### Example 4

Leber congenital amaurosis (LCA) is a debilitating eye disorder and is considered one of the most severe forms of retinal degeneration. Mutations in CEP290 (LCA10) account for >26% of all LCA cases and are the most frequent cause of LCA. Adeno-associated viral (AAV) vectors are currently the most efficient vectors for gene delivery to the retina. However, the development of a gene therapy for LCA10 has been challenging because the size of the CEP290 gene is too large to be packaged into conventional AAV vectors. Mutation specific anti-sense oligo and gene editing therapies have been previously reported.

This example describes a mutation-independent gene therapy delivered with an AAV vector to treat LCA10, that results in severe vision loss at infancy. Versions of CEP290 minigene constructs (miniCEP290) that are functional and can be delivered into the subretinal space using AAV vectors were produced.

CEP290 minigene [e.g., CEP290 amino acid 580-1180 domain] under the control of a ubiquitous promoter was observed to improve the function and survival of photoreceptors in neonatal Cep290-mutant mice (Cep290^{rd16}). However, the effect was short-lived with degeneration ensuing after 5 weeks of age. Data indicate that the expression of CEP290-580-1180 under the control of the photoreceptor-specific rhodopsin kinase promoter improved the electroretinogram (ERG) response for both rod and cone photoreceptors by ˜1.5 folds. This example describes a miniCEP290 gene construct that encodes amino acids 1-200 and 580-1180 (miniCEP290-1-200/580-1180) which improved photoreceptor structural and functional rescue by ˜500% when delivered at postnatal day 10 in Cep290^{rd16} mice. Data also indicate that the expression of the new miniCEP290 prolonged the survival and improved the protein trafficking defects in the photoreceptors in the Cep290rd16 mice.

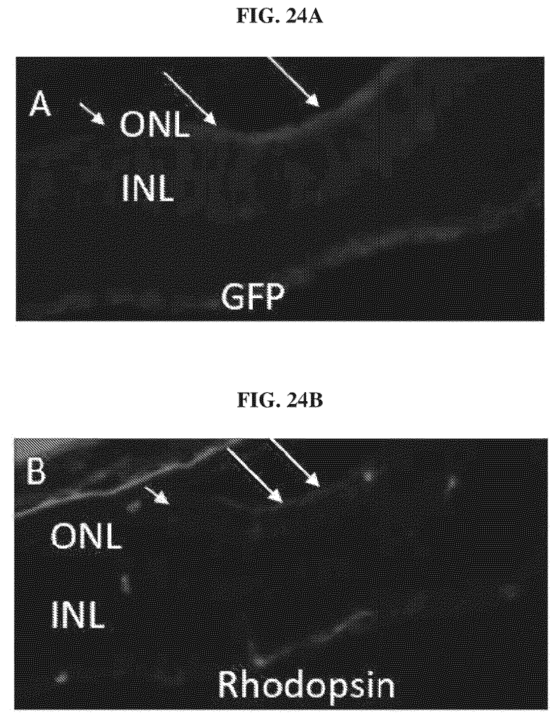

FIGS. 24A-24C show a morphological analysis of Rhodopsin Kinase (RK) promoter driving miniCEP290-580-1180: the minigene was subretinally delivered with AAV8 at P10 stage. The analysis was performed at 9 weeks of age. FIG. 24A shows miniCEP290-580-1180 immunofluorescence analysis of the injected retinal region (GFP; longer arrows). FIG. 24B shows improvement in rhodopsin (longer arrows). Shorter arrows do not show GFP expression (non-transduced) and consequently exhibit undetectable rhodopsin expression. FIG. 24C shows nuclear staining also shows more nuclear layers in the outer nuclear layer (ONL) region in the transduced area (longer arrows and longer vertical bars) as compared to the untransduced region (shorter arrow and shorter vertical bar). Rescue effect lasted more than 14 weeks post-injection.

### Example 5

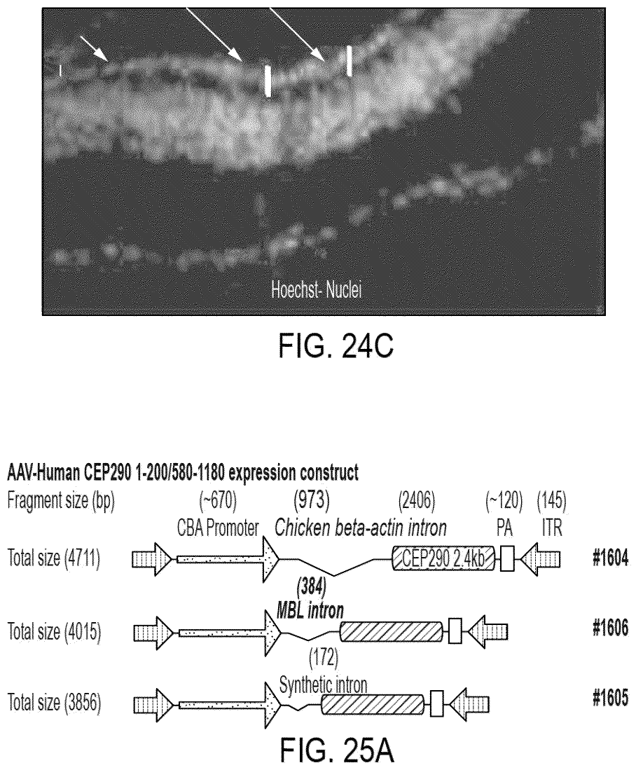

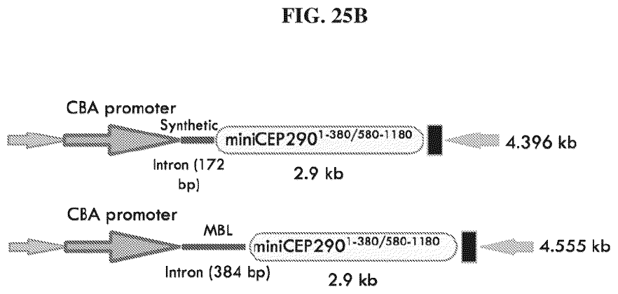

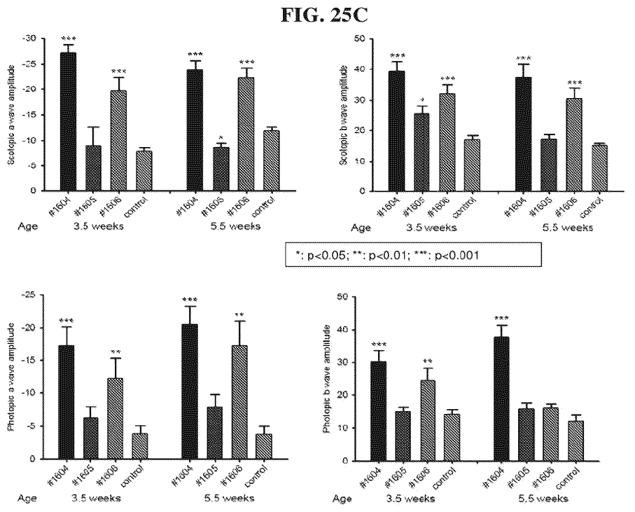

Codon-optimized MiniCEP290 constructs were produced and packaged into rAAVs using AAV5 capsid proteins. The sequences were codon-optmized to increase protein production, reduce tandem rare codons (which can reduce the efficiency of translation or even disengage the translational machinery), prolong the half-life of the mRNA, and to break stem-loop structures. FIGS. 25A-25C show codon optimized miniCEP290 constructs. FIG. 25A shows codon optimized miniCEP290 1-200/580-1180 constructs with promoters (e.g., chicken beta-actin promoter) and different introns (chicken beta-actin intron, synthetic intron, MBL intron, etc.). FIG. 25B shows codon optimized miniCEP290 1-380/580-1180 constructs with different promoters and introns. FIG. 25C shows representative data for codon optimized miniCEP290 1-200/580-1180 constructs packaged into AAV5 capsid and injected subretinally into mice at P10 stage. The mice were then analyzed by ERG (both scotopic and photopic) at the ages indicated.

### Equivalents

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1-43. (canceled)

44. A method for treating an ocular ciliopathy in a subject in need thereof, the method comprising administering to a subject having an ocular ciliopathy a therapeutically effective amount of an isolated nucleic acid,
comprising a transgene encoding a CEP290 fragment having the amino acid sequence set forth in SEQ ID NO: 19 operably linked to a promoter, wherein the promoter is a retinoschisin promoter, K12 promoter, a rhodopsin promoter, a rhodopsin kinase promoter, or an interphotoreceptor retinoid-binding protein proximal (IRBP) promoter, optionally wherein the rhodopsin kinase promoter is a GRK1 promoter.

45. The method of claim 44, wherein the ocular ciliopathy is associated with a mutation of the CEP290 gene in the subject or a deletion of the CEP290 gene in the subject.

46. The method of claim 44, wherein the mutation or deletion of CEP290 results in retinal degeneration, photoreceptor degeneration, retinal dysfunction, retinal pigmented epithelium degeneration/dysfunction, and/or loss of vision.

47. The method of claim 44, wherein the ocular ciliopathy is Leber congenital amaurosis (LCA), Joubert syndrome, Bardet-Biedl syndrome, Meckel syndrome, Usher syndrome, Nephronophthisis, or Senior-Løken syndrome.

48. The method of claim 47, wherein the ocular ciliopathy is Leber congenital amaurosis (LCA).

49. The method of claim 44, wherein the LCA is LCA10.

50. The method of claim 45, wherein the mutation in the CEP290 gene is an intronic mutation, a nonsense mutation, a frameshift mutation, a missense mutation, or any combination thereof.

51. The method of claim 50, wherein the subject is human and the mutation occurs at position c.2991+1655, optionally wherein the mutation is A1655G.

52. The method of claim 44, wherein the administration results in delivery of the isolated nucleic acid or recombinant adeno-associated virus (rAAV) to the eye of the subject.

53. The method of claim 44, wherein the administration is via injection.

54. The method of claim 44, wherein the administration is subretinal administration to the eye of the subject.

55. The method of claim 44, wherein the effective amount results in improvement in photoreceptor (PR) function compared to PR function before the administration.

56. The method of claim 55, wherein the improvement in PR function is measured by ERG.

57. The method of claim 56, wherein the ERG comprises scotopic or photopic measurements.

58. A method for treating an ocular ciliopathy in a subject in need thereof, the method comprising administering to a subject having an ocular ciliopathy a therapeutically effective amount of an rAAV,
wherein the rAAV comprises:
(i) a capsid protein; and,
(ii) an isolated nucleic acid comprising a transgene encoding a CEP290 fragment having the amino acid sequence set forth in SEQ ID NO: 19 operably linked to a promoter, wherein the promoter is a retinoschisin promoter, K12 promoter, a rhodopsin promoter, a rhodopsin kinase promoter, or an interphotoreceptor retinoid-binding protein proximal (IRBP) promoter, optionally wherein the rhodopsin kinase promoter is a GRK1 promoter.


SEQUENCE LISTING (39 sequences)

SEQ ID NO: 1 - protein, 2479 aa, Homo sapiens
MPPNINWKEIMKVDPDDLPRQEELADNLLISLSKVEVNELKSEKQENVIHLFRITQSLMKMKAQEVELALEEVEKAGEEQAKFENQLKTKVMKLENELEMAQQSAGGRDTRFLRNEICQLEKQLEQKDRELEDMEKELEKEKKVNEQLALRNEEAENENSKLRRENKRLKKKNEQLCQDIIDYQKQIDSQKETLLSRRGEDSDYRSQLSKKNYELIQYLDEIQTLTEANEKIEVQNQEMRKNLEESVQEMEKMTDEYNRMKAIVHQTDNVIDQLKKENDHYQLQVQELTDLLKSKNEEDDPIMVAVNAKVEEWKLILSSKDDEIIEYQQMLHNLREKLKNAQLDADKSNVMALQQGIQERDSQIKMLTEQVEQYTKEMEKNTCIIEDLKNELQRNKGASTLSQQTHMKIQSTLDILKEKTKEAERTAELAEADAREKDKELVEALKRLKDYESGVYGLEDAVVEIKNCKNQIKIRDREIEILTKEINKLELKISDFLDENEALRERVGLEPKTMIDLTEFRNSKHLKQQQYRAENQILLKEIESLEEERLDLKKKIRQMAQERGKRSATSGLTTEDLNLTENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAELTKINLDAQKVEQMLRDELADSVSKAVSDADRQRILELEKNEMELKVEVSKLREISDIARRQVEILNAQQQSRDKEVESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLDQSQKESQCLKSELQAQKEANSRAPTTTMRNLVERLKSQLALKEKQQKALSRALLELRAEMTAAAEERIISATSQKEAHLNVQQIVDRHTRELKTQVEDLNENLLKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKTTGMTVDQVLGIRALESEKELEELKKRNLDLENDILYMRAHQALPRDSVVEDLHLQNRYLQEKLHALEKQFSKDTYSKPSISGIESDDHCQREQELQKENLKLSSENIELKFQLEQANKDLPRLKNQVRDLKEMCEFLKKEKAEVQRKLGHVRGSGRSGKTIPELEKTIGLMKKVVEKVQRENEQLKKASGILTSEKMANIEQENEKLKAELEKLKAHLGHQLSMHYESKTKGTEKIIAENERLRKELKKETDAAEKLRIAKNNLEILNEKMTVQLEETGKRLQFAESRGPQLEGADSKSWKSIVVTRMYETKLKELETDIAKKNQSITDLKQLVKEATEREQKVNKYNEDLEQQIKILKHVPEGAETEQGLKRELQVLRLANHQLDKEKAELIHQIEANKDQSGAESTIPDADQLKEKIKDLETQLKMSDLEKQHLKEEIKKLKKELENFDPSFFEEIEDLKYNYKEEVKKNILLEEKVKKLSEQLGVELTSPVAASEEFEDEEESPVNFPIY

SEQ ID NO: 2 - protein, 1116 aa, Homo sapiens
TENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAELTKINLDAQKVEQMLRDELADSVSKAVSDADRQRILELEKNEMELKVEVSKLREISDIARRQVEILNAQQQSRDKEVESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLD

SEQ ID NO: 3 - protein, 601 aa, Homo sapiens
TENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAELTKINLDAQKVEQMLRDELADSVSKAVSDADRQRILELEKNEMELKVEVSKLREISDIARRQVEILNAQQQSRDKEV

SEQ ID NO: 4 - protein, 515 aa, Homo sapiens
ESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLD

SEQ ID NO: 5 - DNA, 3606 bp, Homo sapiens
  source 1..3606: mol_type = genomic DNA
actgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagta

SEQ ID NO: 6 - DNA, 1803 bp, Homo sapiens
  source 1..1803: mol_type = genomic DNA
actgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagta

SEQ ID NO: 7 - DNA, 1545 bp, Homo sapiens
  source 1..1545: mol_type = genomic DNA
gagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggac

SEQ ID NO: 8 - DNA, 7972 bp, Homo sapiens
  source 1..7972: mol_type = genomic DNA
atttgaagtcctcgttccacgccttctcatcatcctgaacaccgagctctgggactccggcggagaatctaaacgtaaagcatcacccacggtcgtgaactgtaggctctcctggcatccgggatcttattctggccttggcggagttggggatggtgtcgcctagcagccgctgccgctttggcttgctcgggaccatttggctggacccagagtccgcgtggaaccgcgatagggatctgtcagggcccgcggccgggtccagcttggtggttgcggtagtgagaggcctccgctggttgccaggcttggtctagaggtggagcacagtgaaagaattcaagatgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtgcaggagcttacagatcttctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagaattagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttactaaaggtcacctataaactttgtttcatttaactatttattaactttataagttaaatatacttggaaataagcagttctccgaactgtagtatttccttctcactaccttgtacctttatacttagattggaattcttaataaataaaattatatgaaattttcaacttattaaaaaaaaaaaaaaaaaa

SEQ ID NO: 9 - protein, 738 aa, unidentified
  REGION 1..738: note = AAV8 capsid protein
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 10 - protein, 1299 aa, Homo sapiens
ESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLDQSQKESQCLKSELQAQKEANSRAPTTTMRNLVERLKSQLALKEKQQKALSRALLELRAEMTAAAEERIISATSQKEAHLNVQQIVDRHTRELKTQVEDLNENLLKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKTTGMTVDQVLGIRALESEKELEELKKRNLDLENDILYMRAHQALPRDSVVEDLHLQNRYLQEKLHALEKQFSKDTYSKPSISGIESDDHCQREQELQKENLKLSSENIELKFQLEQANKDLPRLKNQVRDLKEMCEFLKKEKAEVQRKLGHVRGSGRSGKTIPELEKTIGLMKKVVEKVQRENEQLKKASGILTSEKMANIEQENEKLKAELEKLKAHLGHQLSMHYESKTKGTEKIIAENERLRKELKKETDAAEKLRIAKNNLEILNEKMTVQLEETGKRLQFAESRGPQLEGADSKSWKSIVVTRMYETKLKELETDIAKKNQSITDLKQLVKEATEREQKVNKYNEDLEQQIKILKHVPEGAETEQGLKRELQVLRLANHQLDKEKAELIHQIEANKDQSGAESTIPDADQLKEKIKDLETQLKMSDLEKQHLKEEIKKLKKELENFDPSFFEEIEDLKYNYKEEVKKNILLEEKVKKLSEQLGVELTSPVAASEEFEDEEESPVNFPIY

SEQ ID NO: 11 - protein, 400 aa, Homo sapiens
ILTKEINKLELKISDFLDENEALRERVGLEPKTMIDLTEFRNSKHLKQQQYRAENQILLKEIESLEEERLDLKKKIRQMAQERGKRSATSGLTTEDLNLTENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILA

SEQ ID NO: 12 - protein, 1029 aa, Homo sapiens
ESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLDTTGMTVDQVLGIRALESEKELEELKKRNLDLENDILYMRAHQALPRDSVVEDLHLQNRYLQEKLHALEKQFSKDTYSKPSISGIESDDHCQREQELQKENLKLSSENIELKFQLEQANKDLPRLKNQVRDLKEMCEFLKKEKAEVQRKLGHVRGSGRSGKTIPELEKTIGLMKKVVEKVQRENEQLKKASGILTSEKMANIEQENEKLKAELEKLKAHLGHQLSMHYESKTKGTEKIIAENERLRKELKKETDAAEKLRIAKNNLEILNEKMTVQLEETGKRLQFAESRGPQLEGADSKSWKSIVVTRMYETKLKELETDIAKKNQSITDLKQLVKEATEREQKVNKYNEDLEQQIKILKHVPEGAETEQGLKRELQVLRLANHQLDKEKAELIHQIEANKDQSGAESTIPDADQLKEKIKDLETQLKMSDLEKQHLKEEIKKLKKELENFDPSFFEEIEDLKYNYKEEVKKNILLEEKVKKLSEQLGVELTSPVAASEEFEDEEESPVNFPIY

SEQ ID NO: 13 - protein, 1281 aa, Homo sapiens
TENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAELTKINLDAQKVEQMLRDELADSVSKAVSDADRQRILELEKNEMELKVEVSKLREISDIARRQVEILNAQQQSRDKEVKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKTTGMTVDQVLGIRALESEKELEELKKRNLDLENDILYMRAHQALPRDSVVEDLHLQNRYLQEKLHALEKQFSKDTYSKPSISGIESDDHCQREQELQKENLKLSSENIELKFQLEQANKDLPRLKNQVRDLKEMCEFLKKEKAEVQRKLGHVRGSGRSGKTIPELEKTIGLMKKVVEKVQRENEQLKKASGILTSEKMANIEQENEKLKAELEKLKAHLGHQLSMHYESKTKGTEKIIAENERLRKELKKETDAAEKLRIAKNNLEILNEKMTVQLEETGKRLQFAESRGPQLEGADSKSWKSIVVTRMYETKLKELETDIAKKNQSITDLKQLVKEATEREQKVNKYNEDLEQQIKILKHVPEGAETEQGLKRELQVLRLANHQLDKEKAELIHQIEANKDQSGAESTIPDADQLKEKIKDLETQLKMSDLEKQHLKEEIKKLKKELENFDPSFFEEIEDLKYNYKEEVKKNILLEEKVKKLSEQLGVELTSPVAASEEFEDEEESPVNFPIY

SEQ ID NO: 14 - protein, 786 aa, Homo sapiens
ESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLDQSQKESQCLKSELQAQKEANSRAPTTTMRNLVERLKSQLALKEKQQKALSRALLELRAEMTAAAEERIISATSQKEAHLNVQQIVDRHTRELKTQVEDLNENLLKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKT

SEQ ID NO: 15 - protein, 914 aa, Homo sapiens
ILTKEINKLELKISDFLDENEALRERVGLEPKTMIDLTEFRNSKHLKQQQYRAENQILLKEIESLEEERLDLKKKIRQMAQERGKRSATSGLTTEDLNLTENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILATTGMTVDQVLGIRALESEKELEELKKRNLDLENDILYMRAHQALPRDSVVEDLHLQNRYLQEKLHALEKQFSKDTYSKPSISGIESDDHCQREQELQKENLKLSSENIELKFQLEQANKDLPRLKNQVRDLKEMCEFLKKEKAEVQRKLGHVRGSGRSGKTIPELEKTIGLMKKVVEKVQRENEQLKKASGILTSEKMANIEQENEKLKAELEKLKAHLGHQLSMHYESKTKGTEKIIAENERLRKELKKETDAAEKLRIAKNNLEILNEKMTVQLEETGKRLQFAESRGPQLEGADSKSWKSIVVTRMYETKLKELETDIAKKNQSITDLKQLVKEATEREQKVNKYNEDLEQQIKILKHVPEGAETEQGLKRELQVLRLANHQLDKEKAELIHQIEANKDQSGAESTIPDADQLKEKIKDLETQLKMSDLEKQHLKEEIKKLKKELENFDPSFFEEIEDLKYNYKEEVKKNILLEEKVKKLSEQLGVELTSPVAASEEFEDEEESPVNFPIY

SEQ ID NO: 16 - protein, 885 aa, Homo sapiens
ILTKEINKLELKISDFLDENEALRERVGLEPKTMIDLTEFRNSKHLKQQQYRAENQILLKEIESLEEERLDLKKKIRQMAQERGKRSATSGLTTEDLNLESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLDQSQKESQCLKSELQAQKEANSRAPTTTMRNLVERLKSQLALKEKQQKALSRALLELRAEMTAAAEERIISATSQKEAHLNVQQIVDRHTRELKTQVEDLNENLLKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKT

SEQ ID NO: 17 - protein, 671 aa, Homo sapiens
ILTKEINKLELKISDFLDENEALRERVGLEPKTMIDLTEFRNSKHLKQQQYRAENQILLKEIESLEEERLDLKKKIRQMAQERGKRSATSGLTTEDLNLTENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAQSQKESQCLKSELQAQKEANSRAPTTTMRNLVERLKSQLALKEKQQKALSRALLELRAEMTAAAEERIISATSQKEAHLNVQQIVDRHTRELKTQVEDLNENLLKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKT

SEQ ID NO: 18 - protein, 572 aa, Homo sapiens
TENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAQSQKESQCLKSELQAQKEANSRAPTTTMRNLVERLKSQLALKEKQQKALSRALLELRAEMTAAAEERIISATSQKEAHLNVQQIVDRHTRELKTQVEDLNENLLKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKT

SEQ ID NO: 19 - protein, 801 aa, Homo sapiens
MPPNINWKEIMKVDPDDLPRQEELADNLLISLSKVEVNELKSEKQENVIHLFRITQSLMKMKAQEVELALEEVEKAGEEQAKFENQLKTKVMKLENELEMAQQSAGGRDTRFLRNEICQLEKQLEQKDRELEDMEKELEKEKKVNEQLALRNEEAENENSKLRRENKRLKKKNEQLCQDIIDYQKQIDSQKETLLSRRGETENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAELTKINLDAQKVEQMLRDELADSVSKAVSDADRQRILELEKNEMELKVEVSKLREISDIARRQVEILNAQQQSRDKEV

SEQ ID NO: 20 - DNA, 3900 bp, Homo sapiens
  source 1..3900: mol_type = genomic DNA
gagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttactaa

SEQ ID NO: 21 - DNA, 1200 bp, Homo sapiens
  source 1..1200: mol_type = genomic DNA
atattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgca

SEQ ID NO: 22 - DNA, 3090 bp, Homo sapiens
  source 1..3090: mol_type = genomic DNA
gagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggacacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttactaa

SEQ ID NO: 23 - DNA, 3846 bp, Homo sapiens
  source 1..3846: mol_type = genomic DNA
actgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttactaa

SEQ ID NO: 24 - DNA, 2358 bp, Homo sapiens
  source 1..2358: mol_type = genomic DNA
gagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaaca

SEQ ID NO: 25 - DNA, 2745 bp, Homo sapiens
  source 1..2745: mol_type = genomic DNA
atattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttactaa

SEQ ID NO: 26 - DNA, 2655 bp, Homo sapiens
  source 1..2655: mol_type = genomic DNA
atattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaaca

SEQ ID NO: 27 - DNA, 2013 bp, Homo sapiens
  source 1..2013: mol_type = genomic DNA
atattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcacagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaaca

SEQ ID NO: 28 - DNA, 1716 bp, Homo sapiens
  source 1..1716: mol_type = genomic DNA
actgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcacagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaaca

SEQ ID NO: 29 - DNA, 2406 bp, Homo sapiens
  source 1..2406: mol_type = genomic DNA
atgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtataa

SEQ ID NO: 30 - protein, 1316 aa, Homo sapiens
KNTCIIEDLKNELQRNKGASTLSQQTHMKIQSTLDILKEKTKEAERTAELAEADAREKDKELVEALKRLKDYESGVYGLEDAVVEIKNCKNQIKIRDREIEILTKEINKLELKISDFLDENEALRERVGLEPKTMIDLTEFRNSKHLKQQQYRAENQILLKEIESLEEERLDLKKKIRQMAQERGKRSATSGLTTEDLNLTENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAELTKINLDAQKVEQMLRDELADSVSKAVSDADRQRILELEKNEMELKVEVSKLREISDIARRQVEILNAQQQSRDKEVESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLD

SEQ ID NO: 31 - protein, 514 aa, Homo sapiens
TTGMTVDQVLGIRALESEKELEELKKRNLDLENDILYMRAHQALPRDSVVEDLHLQNRYLQEKLHALEKQFSKDTYSKPSISGIESDDHCQREQELQKENLKLSSENIELKFQLEQANKDLPRLKNQVRDLKEMCEFLKKEKAEVQRKLGHVRGSGRSGKTIPELEKTIGLMKKVVEKVQRENEQLKKASGILTSEKMANIEQENEKLKAELEKLKAHLGHQLSMHYESKTKGTEKIIAENERLRKELKKETDAAEKLRIAKNNLEILNEKMTVQLEETGKRLQFAESRGPQLEGADSKSWKSIVVTRMYETKLKELETDIAKKNQSITDLKQLVKEATEREQKVNKYNEDLEQQIKILKHVPEGAETEQGLKRELQVLRLANHQLDKEKAELIHQIEANKDQSGAESTIPDADQLKEKIKDLETQLKMSDLEKQHLKEEIKKLKKELENFDPSFFEEIEDLKYNYKEEVKKNILLEEKVKKLSEQLGVELTSPVAASEEFEDEEESPVNFPIY

SEQ ID NO: 32 - DNA, 1545 bp, Homo sapiens
  source 1..1545: mol_type = genomic DNA
acaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttactaa

SEQ ID NO: 33 - DNA, 3948 bp, Homo sapiens
  source 1..3948: mol_type = genomic DNA
aagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagaattagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggac

SEQ ID NO: 34 - DNA, 2406 bp, Homo sapiens; identical to SEQ ID NO: 29
  source 1..2406: mol_type = genomic DNA

SEQ ID NO: 35 - DNA, 2946 bp, Homo sapiens
  source 1..2946: mol_type = genomic DNA
atgcccccaaacatcaactggaaagagattatgaaagtggaccctgacgacctgcctagacaggaagaactggctgacaacctgctgatcagcctgtccaaggtggaagtgaatgagctgaagtctgagaagcaggagaacgtgatccacctgttcagaatcacccagagcctgatgaagatgaaggcacaggaggtggagctggccctggaggaggtggagaaggcaggagaggagcaggccaagttcgagaatcagctgaagaccaaagtgatgaagctggagaacgagctggagatggcacagcagtccgccggaggcagggatacacgctttctgcggaacgagatctgccagctggagaagcagctggagcagaaggacagggagctggaggatatggagaaggagctggagaaggagaagaaggtgaacgagcagctggccctgcgcaatgaggaggccgagaatgagaatagcaagctgcggagagagaacaagcggctgaagaagaagaacgagcagctgtgccaggacatcatcgattaccagaagcagatcgactcccagaaggagacactgctgagcaggaggggagaggactccgattatcgcagccagctgtccaagaagaattacgagctgatccagtatctggatgagatccagaccctgacagaggccaacgagaagatcgaggtgcagaaccaggagatgagaaagaatctggaggagtccgtgcaggagatggagaagatgaccgacgagtacaacaggatgaaggccatcgtgcaccagacagacaatgtgatcgatcagctgaagaaggagaacgaccactatcagctgcaggtgcaggagctgaccgatctgctgaagtccaagaatgaggaggacgatcccatcatggtggccgtgaacgccaaggtggaggagtggaagctgatcctgagctccaaggacgatgagatcatcgagtaccagcagatgctgcacaacctgcgcgagaagctgaagaatgcccagctggacgccgataagagcaatgtgatggcactgcagcagggaatccaggagagggactcccagatcaagatgctgaccgagcaggtggagcagtataccaaggagatggagaagacagagaacatctctcagggcgacagaatcagcgagaggaagctggatctgctgtccctgaagaacatgtccgaggcccagtctaagaatgagttcctgtcccgcgagctgatcgagaaggagcgggacctggagcggagccggacagtgatcgccaagtttcagaataagctgaaggagctggtggaggagaacaagcagctggaggagggcatgaaggagatcctgcaggccatcaaggagatgcagaaggacccagatgtgaagggcggcgagacaagcctgatcatcccctctctggagcgcctggtgaacgccatcgagagcaagaatgccgagggcatctttgacgcctccctgcacctgaaggcccaggtggatcagctgacaggccgcaatgaggagctgcggcaggagctgagagagtctaggaaggaggccatcaattacagccagcagctggccaaggccaacctgaagatcgaccacctggagaaggagacaagcctgctgcggcagtctgagggcagcaacgtggtgttcaagggaatcgacctgccagatggaatcgcaccttctagcgccagcatcatcaactcccagaatgagtacctgatccacctgctgcaggagctggagaacaaggagaagaagctgaagaatctggaggactctctggaggattataatagaaagtttgccgtgatcaggcaccagcagtccctgctgtacaaggagtatctgtctgagaaggagacatggaagacagagagcaagaccatcaaggaggagaagaggaagctggaggaccaggtgcagcaggatgccatcaaggtgaaggagtacaacaatctgctgaacgccctgcagatggactctgatgagatgaagaagatcctggccgagaatagcagaaagatcacagtgctgcaggtgaacgagaagtccctgatcaggcagtataccacactggtggagctggagcgccagctgcggaaggagaacgagaagcagaagaatgagctgctgtctatggaggccgaggtgtgcgagaagatcggctgtctgcagagattcaaggagatggccatctttaagatcgccgccctgcagaaggtggtggacaattctgtgagcctgtccgagctggagctggccaacaagcagtacaatgagctgaccgccaagtatcgcgacatcctgcagaaggataacatgctggtgcagcggacaagcaatctggagcacctggagtgcgagaacatcagcctgaaggagcaggtggagtccatcaacaaggagctggagatcaccaaggagaagctgcacacaatcgagcaggcctgggagcaggagacaaagctgggcaatgagtcctctatggacaaggccaagaagtctatcaccaacagcgatatcgtgtctatcagcaagaagatcacaatgctggagatgaaggagctgaatgagaggcagcgcgccgagcactgtcagaagatgtacgagcacctgagaacatccctgaagcagatggaggagaggaatttcgagctggagacaaagtttgccgagctgacaaagatcaacctggacgcccagaaggtggagcagatgctgagagacgagctggccgattccgtgtctaaggccgtgagcgacgccgatcggcagagaatcctggagctggagaagaacgagatggagctgaaggtggaggtgtccaagctgagggagatctctgatatcgctcggcggcaggtggagattctgaacgcacagcagcagtcacgggacaaggaagtctga

SEQ ID NO: 36 - protein, 981 aa, Homo sapiens
MPPNINWKEIMKVDPDDLPRQEELADNLLISLSKVEVNELKSEKQENVIHLFRITQSLMKMKAQEVELALEEVEKAGEEQAKFENQLKTKVMKLENELEMAQQSAGGRDTRFLRNEICQLEKQLEQKDRELEDMEKELEKEKKVNEQLALRNEEAENENSKLRRENKRLKKKNEQLCQDIIDYQKQIDSQKETLLSRRGEDSDYRSQLSKKNYELIQYLDEIQTLTEANEKIEVQNQEMRKNLEESVQEMEKMTDEYNRMKAIVHQTDNVIDQLKKENDHYQLQVQELTDLLKSKNEEDDPIMVAVNAKVEEWKLILSSKDDEIIEYQQMLHNLREKLKNAQLDADKSNVMALQQGIQERDSQIKMLTEQVEQYTKEMEKTENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAELTKINLDAQKVEQMLRDELADSVSKAVSDADRQRILELEKNEMELKVEVSKLREISDIARRQVEILNAQQQSRDKEV

SEQ ID NO: 37 - DNA, 973 bp, Gallus gallus
  source 1..973: mol_type = genomic DNA
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 38 - DNA, 172 bp, synthetic construct
  misc_feature 1..172: note = Synthetic Polynucleotide
  source 1..172: mol_type = other DNA
gaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtaccc

SEQ ID NO: 39 - DNA, 384 bp, unidentified
  misc_feature 1..384: note = MBL intron
  source 1..384: mol_type = genomic DNA
tcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgtttgctgcccgtgaccagcacgtcaacgattttgtgggcacgggcgacaccgcagtgtagtctgagcagtactcgttgctgccgcgcgcgccaccagacataatagctgacagactaacagactgttcctttccatgggtcttttctgcagtcaccgtcgccgc